(19) United States
(12) Patent Application Publication
(10) Pub. No.: US 2013/0203706 A1
(43) Pub. Date: Aug. 8, 2013

(54) COMPOUNDS AND METHODS FOR THE TREATMENT OR PREVENTION OF FLAVIVIRUS INFECTIONS

(71) Applicant: Vertex Pharmaceuticals Incorporated, Cambridge, MA (US)

(72) Inventor: Jeremy Green, Waltham, MA (US)

(73) Assignee: Vertex Pharmaceuticals Incorporated, Cambridge, MA (US)

(21) Appl. No.: 13/724,014

(22) Filed: Dec. 21, 2012

Related U.S. Application Data
(63) Continuation of application No. PCT/US2011/042119, filed on Jun. 28, 2011.

(51) Int. Cl. C07D 333/38; C07F 9/6553

(52) U.S. Cl. CPC C07D 333/38; C07F 9/655345
USPC 514/95; 549/69; 514/447; 549/8; 435/184

(57) ABSTRACT

A compound is represented by Structural Formula (I):

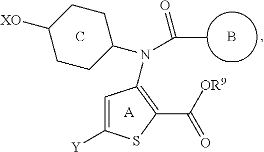

or a pharmaceutically acceptable salt thereof, wherein the variables of Structural Formula (I) are as described in the specification and the claims. A pharmaceutical composition comprises a compound represented by Structural Formula (I) or a pharmaceutically acceptable salt thereof, and a pharmaceutically acceptable carrier of excipient. A method of treating a HCV infection in a subject comprises administering to the subject a therapeutically effective amount of a compound represented by Structural Formula (I) or a pharmaceutically acceptable salt thereof. A method of inhibiting or reducing the activity of HCV polymerase in a subject or in a biological in vitro sample comprises administering to the subject or to the sample a therapeutically effective amount of a compound represented by Structural Formula (I) or a pharmaceutically acceptable salt thereof.

### RELATED APPLICATIONS

This application is a continuation of PCT Application Number PCT/US2011/042119, filed Jun. 28, 2011, which claims priority to U.S. Provisional Applications, U.S. Ser. No. 61/359,164 filed on Jun. 28, 2010 and to U.S. Ser. No. 61/436,649 filed on Jan. 27, 2011. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flavivirus infections.

### SUMMARY OF THE INVENTION

The present invention generally relates to compounds useful for treating or preventing Flavivirus infections, such as HCV infections.

In one embodiment, the invention is directed to a compound represented by Structural Formula (I):

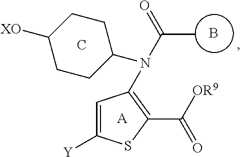

or a pharmaceutically acceptable salt thereof, wherein:

Ring A is optionally further substituted with one or more substituents selected from the group consisting of halogen, -D, —CD₃, —CHD₂, —CH₂D, —CN, C₁₋₄ alkyl, and C₁₋₄ haloalkyl.

Ring B is a C₃₋₈ cycloalkyl ring or a 5-6 membered aryl ring, each of which optionally and independently is substituted with one or more substituents selected from the group consisting of halogen, -D (deuterium), —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂.

Ring C is a cyclohexyl ring optionally further substituted with one or more substituents selected from the group consisting of halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂.

X is [—C(O)—C(R⁴R⁵)—N(R)—]ₙ—C(O)C(R⁶R⁷R⁸), —P(O)(OR³)₂, or —C(O)R².

Y is C₃₋₈ carbocycle, 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y} independently selected from the group consisting of halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}, and wherein said C₂ aliphatic group is optionally substituted with one or more substitutents selected from the group consisting of halogen, —CN, C₁₋₂ alkyl, C₁₋₂ haloalkyl, hydroxy, and methoxy.

R¹ is —H or a C₁₋₆ alkyl, C₃₋₁₀ carbocyclic, 4-10 membered heterocyclic, C₆₋₁₀ aryl, or 5-10 membered heteroaryl group, wherein said alkyl group is optionally substituted with one or more instances of J^{1A}, and wherein each of said carbocyclic and heterocyclic groups is optionally and independently substituted with one or more instances of J^{1B}, and wherein each of said aryl and heteroaryl groups is optionally and independently substituted with one or more instances of J^{1C}.

R² is a C₃₋₈ carbocyclic, 3-8 membered heterocyclic, C₆₋₁₀ aryl, or 5-10 membered heteroaryl group, wherein each of said carbocyclic and heterocyclic groups is independently and optionally substituted with one or more instances of J^{E}, and each of said aryl and heteroaryl groups is independently and optionally substituted with one or more instances of J^{F}.

R³ is —H, a C₁₋₆ aliphatic, C₃₋₈ carbocyclic, 3-8 membered heterocyclic, C₆₋₁₀ aryl, or 5-10 membered heteroaryl group, wherein said aliphatic group is optionally substituted with one or more instances of J^{D}, each of said carbocyclic and heterocyclic groups is independently and optionally substituted with one or more instances of J^{E}, and each of said aryl and heteroaryl groups is independently and optionally substituted with one or more instances of J^{F}.

Each of R⁴, R⁵, R⁶, and R⁷ independently is —H; or C₁₋₆ alkyl optionally substituted with one or more substitutents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O) N(C₁₋₆ alkyl)₂, —NHC(O)(C₁₋₆ alkyl), phenyl, hydroxyphenyl, imidazole, and indole.

R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}; or

optionally when R⁸ is —NR^{b}R^{c}, R^{c} and R⁷ form a pyrrolidine ring

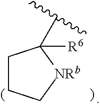

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}.

Each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E}.

Each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E}.

Each of J^{1C} and J^{9C} independently is Q or a C₁₋₆ aliphatic group optionally substituted with one or more instances of Q; or two J^{1C} and two J^{9C}, respectively, together with the atoms to which they are attached, optionally and independently form a 3-8-membered non-aromatic ring that is optionally substituted with one or more instances of J^{E}.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}.

Each R^{a}, R^{b}, and R^{c} independently is: i) —H; ii) a C₁₋₆ aliphatic group optionally substituted with one or more substituents independently selected from the group consisting of halogen, oxo, nitro, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or

R^{a}, together with R and the nitrogen atom to which it is attached, optionally forms a 4-8 membered heterocycle optionally substituted with one or more instances of J^{E}; or

R^{b} and R^{c}, together with the nitrogen atom to which they are attached, optionally forms a 4-8 membered heterocycle optionally substituted with one or more instances of J^{E}.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or two R′, together with the nitrogen atom to which they are attached, optionally form a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

Each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

In another embodiment, the invention is directed to a compound represented by Structural Formula (II):

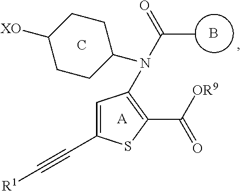

or a pharmaceutically acceptable salt thereof, wherein:

Ring A is optionally further substituted with one or more substituents selected from the group consisting of halogen, -D, —CD₃, —CHD₂, —CH₂D, —CN, C₁₋₄ alkyl, and C₁₋₄ haloalkyl.

Ring B is a C₃₋₈ cycloalkyl ring or a 5-6 membered aryl ring, each of which optionally and independently is substituted with one or more substituents selected from the group consisting of halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂.

Ring C is a cyclohexyl ring optionally further substituted with one or more substituents selected from the group consisting of halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂.

X is [—C(O)—C(R⁴R⁵)—N(R)—]ₙ—C(O)C(R⁶R⁷R⁸), —P(O)(OR³)₂, or —C(O)R².

R¹ is —H or a C₁₋₆ alkyl, C₃₋₁₀ carbocyclic, 4-10 membered heterocyclic, C₆₋₁₀ aryl, or 5-10 membered heteroaryl group, wherein said alkyl group is optionally substituted with one or more instances of J^{1A}, and wherein each of said carbocyclic and heterocyclic groups is optionally and independently substituted with one or more instances of J^{1B}, and wherein each of said aryl and heteroaryl groups is optionally and independently substituted with one or more instances of J^{1C}.

R² is a C₃₋₈ carbocyclic, 3-8 membered heterocyclic, C₆₋₁₀ aryl, or 5-10 membered heteroaryl group, wherein each of said carbocyclic and heterocyclic groups is independently and optionally substituted with one or more instances of J^{E}, and each of said aryl and heteroaryl groups is independently and optionally substituted with one or more instances of J^{F}.

R³ is —H, a C₁₋₆ aliphatic, C₃₋₈ carbocyclic, 3-8 membered heterocyclic, C₆₋₁₀ aryl, or 5-10 membered heteroaryl group, wherein said aliphatic group is optionally substituted with one or more instances of J^{D}, each of said carbocyclic and heterocyclic groups is independently and optionally substituted with one or more instances of J^{E}, and each of said aryl and heteroaryl groups is independently and optionally substituted with one or more instances of J^{F}.

Each of R⁴, R⁵, R⁶, and R⁷ independently is —H; or C₁₋₆ alkyl optionally substituted with one or more substituents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O)N(C₁₋₆ alkyl)₂, —NHC(O)(C₁₋₆ alkyl), phenyl, hydroxyphenyl, imidazole, and indole.

R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}; or optionally when R⁸ is —NR^{b}R^{c}, R^{c} and R⁷ form a pyrrolidine ring

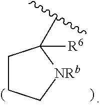

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}.

Each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E}.

Each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E}.

Each of J^{1C} and J^{9C} independently is Q or a C₁₋₆ aliphatic group optionally substituted with one or more instances of Q; or two J^{1C} and two J^{9C}, respectively, together with the atoms to which they are attached, optionally and independently form a 3-8-membered non-aromatic ring that is optionally substituted with one or more instances of J^{E}.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}.

Each R^{a}, R^{b}, and R^{c} independently is: i) —H; ii) a C₁₋₆ aliphatic group optionally substituted with one or more substituents independently selected from the group consisting of halogen, oxo, nitro, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or

R^{a}, together with R and the nitrogen atom to which it is attached, optionally forms a 4-8 membered heterocycle optionally substituted with one or more instances of J^{E}; or

R^{b} and R^{c}, together with the nitrogen atom to which they are attached, optionally forms a 4-8 membered heterocycle optionally substituted with one or more instances of J^{E}.

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or two R′, together with the nitrogen atom to which they are attached, optionally form a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

Each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1.

In a specific embodiment, the invention is directed to a compound represented by any one of Structural Formula (I) or (II), or a pharmaceutically acceptable salt thereof, wherein the values of the variables of each of Structural Formulae (I) and (II) are each and independently as described above except that R⁴ and R⁶ are each independently —H or C₁₋₆ alkyl.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that R⁴ and R⁶ are each independently —H.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

R⁴ and R⁶ are each independently —H or C₁₋₆ alkyl;

X is [—C(O)—C(R⁴R⁵)—N(R)—]ₙ—C(O)C(R⁶R⁷R⁸) or —P(O)(OR³)₂; and

R⁸ is —NR^{b}R^{c}.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

X is [—C(O)—C(R⁴R⁵)—N(R)—]ₙ—C(O)C(R⁶R⁷R⁸) or —P(O)(OR³)₂;

each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl; or alternatively, each R³ independently is —H or substituted C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; or alternatively each R³ independently is —H or C₁₋₆ alkyl;

R⁴ and R⁶ are each independently —H or C₁₋₆ alkyl; and

R⁸ is —NR^{b}R^{c}.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

R⁴ and R⁶ are each independently —H or C₁₋₆ alkyl; and

R⁵ and R⁷ are each independently —H or optionally substituted C₁₋₆ alkyl.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

R⁴ and R⁶ are each independently —H; and

R⁵ and R⁷ are each independently —H or optionally substituted C₁₋₆ alkyl.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

R⁴ and R⁶ are each independently —H or C₁₋₆ alkyl;

R⁵ and R⁷ are each independently —H or optionally substituted C₁₋₆ alkyl; and

X is [—C(O)—C(R⁴R⁵)—N(R)—]ₙ—C(O)C(R⁶R⁷R⁸) or —P(O)(OR³)₂.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

X is [—C(O)—C(R⁴R⁵)—N(R)—]ₙ—C(O)C(R⁶R⁷R⁸) or —P(O)(OR³)₂;

each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl; or alternatively, each R³ independently is —H or substituted C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; or alternatively each R³ independently is —H or C₁₋₆ alkyl; and

R⁴ and R⁶ are each independently —H or C₁₋₆ alkyl;

R⁵ and R⁷ are each independently —H or optionally substituted C₁₋₆ alkyl;

X is [—C(O)—C(R⁴R⁵)—N(R)—]ₙ—C(O)C(R⁶R⁷R⁸) or —P(O)(OR³)₂; and

R⁸ is —NR^{b}R^{c}.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that:

R⁴ and R⁶ are each independently —H or C₁₋₆ alkyl;

R⁵ and R⁷ are each independently —H or optionally substituted C₁₋₆ alkyl;

X is [—C(O)—C(R⁴R⁵)—N(R)—]ₙ—C(O)C(R⁶R⁷R⁸) or —P(O)(OR³)₂; and

R⁸ is —NR^{b}R^{c}.

In a specific embodiment, the invention is directed to a compound represented by Structural Formula (I) or (II), or a pharmaceutically acceptable salt thereof, wherein the values of the variables of each of Structural Formulae (I) and (II) are each and independently as described above except that n is 0.

In another embodiment, the invention is directed to a pharmaceutical composition comprising a compound described herein (e.g., a compound of the invention described in the claims and FIGS. 1 and 2, such as a compound represented by any one of Structural Formulae (I)-(VII) or a pharmaceutically acceptable salt thereof) and a pharmaceutically acceptable carrier or excipient.

In yet another embodiment, the invention provides methods of treating a HCV infection in a subject, comprising administering to the subject a therapeutically effective amount of a compound described herein (e.g., a compound of the invention described in the claims and FIGS. 1 and 2, such as a compound represented by any one of Structural Formulae (I)-(VII) or a pharmaceutically acceptable salt thereof).

In yet another embodiment, the invention is directed to a method of inhibiting or reducing the activity of HCV polymerase in a subject, comprising administering to the subject a therapeutically effective amount of a compound described herein (e.g., a compound of the invention described in the claims and FIGS. 1 and 2, such as a compound represented by any one of Structural Formulae (I)-(VII) or a pharmaceutically acceptable salt thereof).

In yet another embodiment, the invention is directed to a method of inhibiting or reducing the activity of HCV polymerase in a biological in vitro sample, comprising administering to the sample an effective amount of a compound of the invention described herein, such as a compound described herein (e.g., a compound of the invention described in the claims and FIGS. 1 and 2, such as a compound represented by any one of Structural Formulae (I)-(VII) or a pharmaceutically acceptable salt thereof).

The present invention also provides use of the compounds of the invention described herein (e.g., the compounds of the invention described in the claims and FIGS. 1 and 2, such as the compounds represented by any one of Structural Formulae (I)-(VII) or pharmaceutically acceptable salts thereof) for the manufacture of the medicament for treating a HCV infection in a subject, or for inhibiting or reducing the activity of HCV polymerase in a subject.

Also provided herein is use of the compounds of the invention described herein (e.g., the compounds of the invention described in the claims and FIGS. 1 and 2, such as the compounds represented by any one of Structural Formulae (I)-(VII) or pharmaceutically acceptable salts thereof) for treating a HCV infection in a subject, or for inhibiting or reducing the activity of HCV polymerase in a subject.

In some embodiments, the compounds of the invention can be employed as prodrugs which generate active metabolites (e.g., the compounds of Structural Formulae (I) or (II) wherein X is —H) useful for treating a HCV infection in a subject, or for inhibiting or reducing the activity of HCV polymerase in a subject (see, for example, Example 2).

### DESCRIPTION OF THE DRAWINGS

FIGS. 1 and 2 show certain compounds of the invention.

FIGS. 3-5 show graphs showing plasma levels of prodrugs of the invention and their conversion into corresponding active metabolites after dosing of the prodrug.

### DETAILED DESCRIPTION OF THE INVENTION

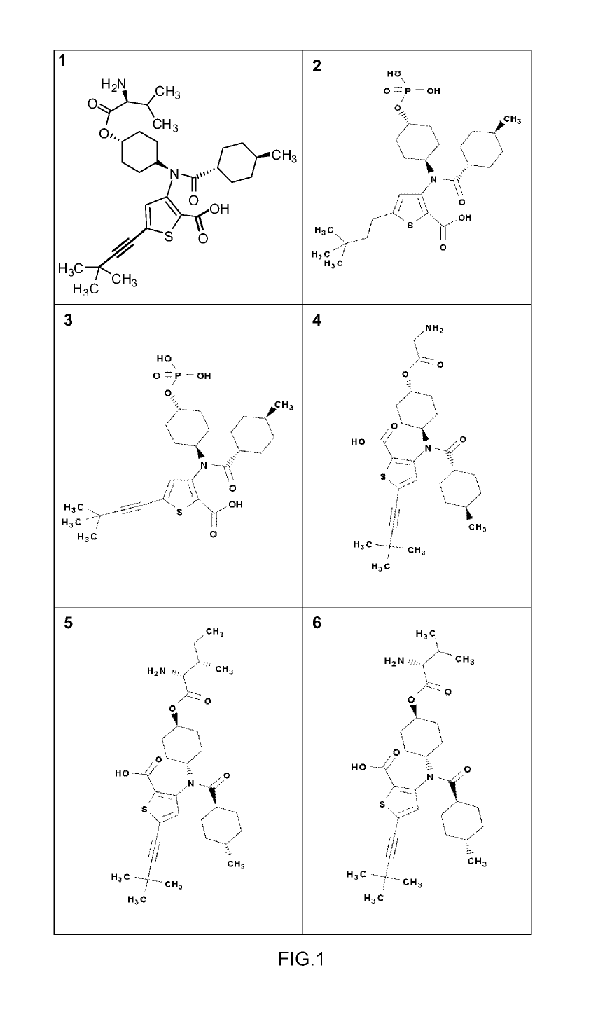

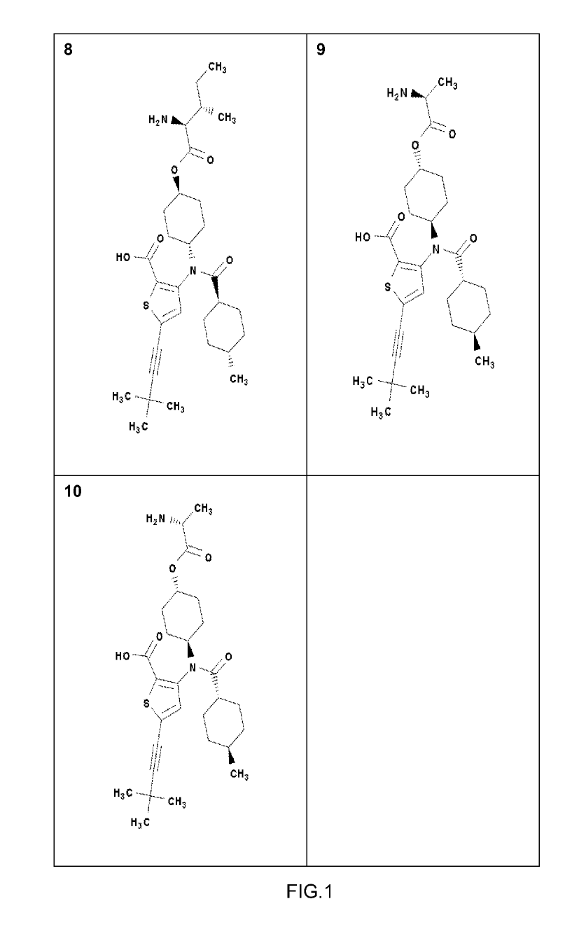

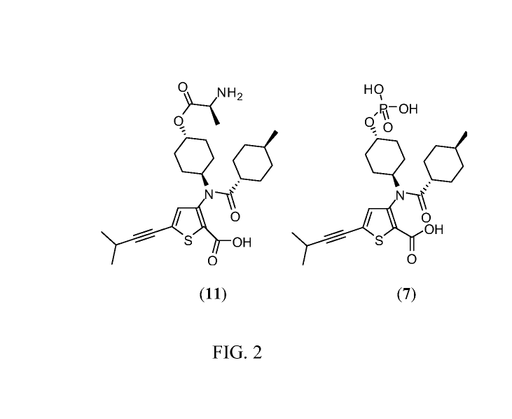

The compounds of the invention are as described in the claims. In some embodiments, the compounds of the invention are represented by any one of Structural Formulae (I)-(VII) or pharmaceutically acceptable salts thereof, wherein the variables are each and independently as described in any one of the claims. In some embodiments, the compounds of the invention are represented by any chemical formulae depicted in FIGS. 1 and 2, or pharmaceutically acceptable salts thereof. In some embodiments, the compounds of the invention are presented by any one of Structural Formulae (I)-(VII) or pharmaceutically acceptable salts thereof, wherein the variables are each and independently as depicted in the chemical formulae in FIGS. 1 and 2.

In one embodiment, the compounds of the invention are represented by Structural Formula (I):

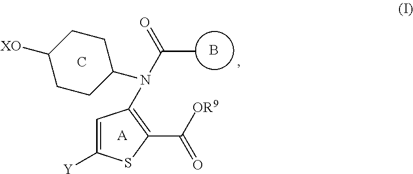

or pharmaceutically acceptable salts thereof. A first set of values of the variables of Structural Formula (I) is as follows:

Ring A is optionally further substituted with one or more substituents. Typical examples of substituents suitable for ring A include halogen, -D, —CD₃, —CHD₂, —CH₂D, —CN, C₁₋₄ alkyl, and C₁₋₄ haloalkyl. A specific example of the substituents includes halogen.

Ring B is an optionally substituted C₃₋₈ cycloalkyl ring or an optionally substituted 5-6 membered aryl ring. In one aspect, ring B is an optionally substituted C₃₋₈ cycloalkyl, or C₅₋₆ cycloalkyl. In another aspect, ring B is an optionally substituted cyclohexyl. Typical substituents suitable for ring B include halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂. Specific examples of substituents suitable for ring B include halogen, -D, —CD₃, —CHD₂, —CH₂D, —CH₃, —CF₃, —OH, —O(CH₃), —CN, —NH₂, —NH(CH₃), and —N(CH₃)₂. More specific examples of substituents suitable for ring B include halogen, —CD₃, —CHD₂, —CH₂D, —CH₃, —CF₃, —OH, and —O(CH₃). More specific examples of substituents suitable for ring B include halogen, C₁₋₄ alkyl, C₁₋₄ haloalkyl, —OH, and —O(C₁₋₄ alkyl).

Ring C is a cyclohexyl ring that is optionally further substituted with one or more substituents. Typical substituents suitable for ring C include halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂. Specific examples of substituents suitable for ring C include halogen, -D, —CD₃, —CHD₂, —CH₂D, —CH₃, —CF₃, —OH, —O(CH₃), —CN, —NH₂, —NH(CH₃), and —N(CH₃)₂. More specific examples of substituents suitable for ring C include halogen, —CD₃, —CHD₂, —CH₂D, —CH₃, —CF₃, —OH, and —O(CH₃). More specific examples of substituents suitable for ring C include halogen, C₁₋₄ alkyl, C₁₋₄ haloalkyl, —OH, and —O(C₁₋₄ alkyl).

X is [—C(O)—C(R⁴R⁵)—N(R)—]ₙ—C(O)C(R⁶R⁷R⁸), —P(O)(OR³)₂, or —C(O)R². In one aspect, X is [—C(O)—C(R⁴R⁵)—N(R)—]ₙ—C(O)C(R⁶R⁷R⁸). In another aspect, X is —C(O)R².

Y is C₃₋₈ carbocycle, optionally substituted 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y}, and wherein said C₂ aliphatic group is optionally substituted with one or more substitutents. In one specific aspect, Y is optionally substituted C₃₋₆ cycloalkyl, optionally substituted C₄₋₆ cycloalkenyl, —(C₂ aliphatic group)-R¹, optionally substituted phenyl, or optionally substituted 5-6 membered heteroaryl, and wherein said C₂ aliphatic group is optionally substituted. In another specific aspect, Y is optionally substituted phenyl, optionally substituted thienyl, or optionally substituted pyridyl. In yet another aspect, Y is optionally substituted phenyl. In yet another specific aspect, Y is optionally substituted C₃₋₆ cycloalkyl or optionally substituted C₄₋₆ cycloalkenyl. In yet another specific aspect, Y is optionally substituted C₄₋₆ cycloalkenyl. In yet another specific aspect, Y is optionally substituted cyclohexenyl. In yet another specific aspect, Y is —(C₂ aliphatic group)-R¹ or phenyl, wherein said C₂ aliphatic group is optionally substituted. In yet another specific aspect, Y is —(C₂ aliphatic group)-R¹, wherein said C₂ aliphatic group is optionally substituted. In yet another specific aspect, Y is —CH₂—CH₂—R¹, —CH═CH—R¹, or —C≡CR¹. In yet another specific aspect, Y is —C≡CR¹.

Typical examples of J^{Y} include halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}. Specific examples of J^{Y} include halogen, —CN, nitro, R^{a}, —OR^{a}, —COR^{a}, and —NRR^{a}. More specific examples of J^{Y} include halogen, —CN, nitro, C₁₋₆ alkyl, C₁₋₆ haloalkyl, —OH, —O(C₁₋₆ alkyl), —O(phenyl), —O(5-6 membered heteroaryl), —NH₂, —NH(C₁₋₆ alkyl), —N(C₁₋₆ alkyl)₂, and —C(O)(C₁₋₆ alkyl). Even more specific examples of J^{Y} include chloro, fluoro, —CN, nitro, methyl, ethyl, —CF₃, —OH, —OMe, —NH₂, and —C(O)Me.

Typical examples of substituents suitable for the C₂ aliphatic group of —(C₂ aliphatic group)-R¹ include halogen, —CN, C₁₋₂ alkyl, C₁₋₂ haloalkyl, hydroxy, and methoxy.

R¹ is i) —H; ii) a C₁₋₆ aliphatic group optionally substituted with one or more instances of J^{1A}; iii) a C₃₋₁₀ carbocycle or 4-10 membered heterocycle, each of which is optionally and independently substituted with one or more instances of J^{1B}; or iv) a C₆₋₁₀ aryl or 5-10 membered heteroaryl group, each of which is optionally and independently substituted with one or more instances of J^{1C}. In one specific aspect, R¹ is an optionally substituted C₁₋₆ alkyl or optionally substituted C₃₋₈ carbocyclic group. In another specific aspect, R¹ is an optionally substituted C₁₋₆ alkyl or optionally substituted C₃₋₈ cycloalkyl group. In yet another specific aspect, R¹ is optionally substituted C₁₋₆ alkyl or optionally substituted C₃-8 cycloalkyl. In yet another aspect, R¹ is C₁₋₆ alkyl or C₃₋₈ cycloalkyl. In yet another specific aspect, R¹ is C₁₋₆ alkyl. In yet another aspect, R¹ is t-butyl or isopropyl.

R² is: i) a C₃₋₁₀ carbocyclic or 4-10 membered heterocyclic group, each of which is optionally and independently substituted with one or more instances of J^{E}, or ii) a C₆₋₁₀ aryl or 5-10 membered heteroaryl group, each of which is optionally and independently substituted with one or more instances of J^{F}. In one specific aspect, R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group. In another specific aspect, R² is an optionally substituted phenyl, or optionally substituted 5-6 membered heteroaryl group.

R³ is: i) —H, ii) a C₁₋₆ aliphatic group optionally substituted with one or more instances of J^{D}, iii) a C₃₋₁₀ carbocyclic or 4-10 membered heterocyclic group, each of which is optionally and independently substituted with one or more instances of J^{E}, or iv) a C₆₋₁₀ aryl or 5-10 membered heteroaryl group, each of which is optionally and independently substituted with one or more instances of J^{F}. In one specific aspect, each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. In another specific aspect, each R³ independently is —H or optionally substituted C₁₋₆ alkyl.

Each of R⁴, R⁵, R⁶, and R⁷ independently is —H; or C₁₋₆ alkyl optionally substituted with one or more substitutents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O)N(C₁₋₆ alkyl)₂, —NHC(O)(C₁₋₆ alkyl), phenyl, hydroxyphenyl, imidazole, and indole. In one specific aspect, each of R⁴, R⁵, R⁶, and R⁷ independently is —H; or C₁₋₆ alkyl optionally substituted with one or more substitutents selected from the group consisting of —OH, —NH₂, —NHC(═NH)NH₂, —CO₂H, —C(O)NH₂, phenyl, hydroxyphenyl, imidazole, and indole. In another specific aspect, R⁴ and R⁶ are each independently —H or C₁₋₆ alkyl; and R⁵ and R⁷ are each independently —H or optionally substituted C₁₋₆ alkyl. In yet another specific aspect, R⁴ and R⁶ are each independently —H or C₁₋₆ alkyl; and R⁵ and R⁷ are each independently —H or C₁₋₆ alkyl optionally substituted with one or more substitutents selected from the group consisting of —OH, —NH₂, —NH—C(═NH)—NH₂, —CO₂H, and —C(O)NH₂. In yet another specific aspect, R⁶ is —H; R⁷ is —H or C₁₋₆ alkyl optionally substituted with one or more substitutents selected from the group consisting of —OH, —NH₂, —NH—C(═NH)—NH₂, —CO₂H, and —C(O)NH₂. In yet another specific aspect, R⁴, R⁵ and R⁶ are each independently —H or C₁₋₆ alkyl; and R⁷ is —H or C₁₋₆ alkyl optionally substituted with one or more substitutents selected from the group consisting of —OH, —NH₂, —NH—C(═NH)—NH₂, —CO₂H, and —C(O)NH₂.

R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}; or optionally when R⁸ is —NR^{b}R^{c}, R^{c} and R⁷ form a pyrrolidine ring. In one specific aspect, R⁸ is —H, halogen, cyano, —OR^{b}, —NR^{b}R^{c} optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. In another specific aspect, R⁸ is —NR^{b}R^{c}.

R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C}. In one specific aspect, R⁹ is —H, or an optionally substituted C₁₋₆ aliphatic or optionally substituted carbocyclic group. In another specific aspect, R⁹ is —H or optionally substituted C₁₋₆ alkyl. In yet another aspect, R⁹ is —H.

Each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E}. In one specific aspect, each of J^{1A} and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl. In another specific aspect, each of J^{1A} and J^{9A} independently is halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), or phenyl. In yet another aspect, each of J^{1A} and J^{9A} independently is halogen, —CN, —OH, —O(C₁₋₆ alkyl), or —O(C₁₋₆ haloalkyl).

Each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E}. In one specific aspect, each of J^{1B} and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl. In another specific aspect, each of J^{1B} and J^{9B} independently is halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); or C₁₋₆ alkyl optionally substituted with one or more substituents selected from the group consisting of halogen, —CN, —OH, —O(C₁₋₆ alkyl), and —O(C₁₋₆ haloalkyl). In yet another specific aspect, each of J^{1B} and J^{9B} independently is halogen, —CN, —OH, —O(C₁₋₆ alkyl), —O(C₁₋₆ haloalkyl), C₁₋₆ alkyl, or C₁₋₆ haloalkyl.

Each of J^{1C} and J^{9C} independently is Q or a C₁₋₆ aliphatic group optionally substituted with one or more instances of Q; or two J^{1C} and two J^{9C}, respectively, together with the atoms to which they are attached, optionally and independently form a 3-8-membered non-aromatic ring that is optionally substituted with one or more instances of J^{E}. In one specific aspect, each of J^{1C} and J^{9C} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl. In another specific aspect, each of J^{1C} and J^{9C} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl. In yet another specific aspect, each of J^{1C} and J^{9C} independently is halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); or C₁₋₆ alkyl optionally substituted with one or more substituents selected from the group consisting of halogen, —CN, —OH, —O(C₁₋₆ alkyl), and —O(C₁₋₆ haloalkyl). In yet another specific aspect, each of J^{1C} and J^{9C} independently is halogen, —CN, —OH, —O(C₁₋₆ alkyl), —O(C₁₋₆ haloalkyl), C₁₋₆ alkyl, or C₁₋₆ haloalkyl.

Each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. Alternatively, each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F}. In one specific aspect, each Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)OR^{a}, —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. In another specific aspect, each Q independently is selected from the group consisting of halogen; cyano; nitro; —OR^{a}; —SR^{a}; —S(O)R^{a}; —SO₂R^{a}; —NRR^{a}, —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —OCONRR^{a}; —C(O)NRC(O)OR^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. In yet another specific aspect, each Q independently is selected from the group consisting of halogen; cyano; nitro; —OR^{a}; —NRR^{a}; —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —OCONRR^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl.

Each R^{a}, R^{b}, and R^{c} independently is: i) —H; ii) a C₁₋₆ aliphatic group optionally substituted with one or more substituents independently selected from the group consisting of halogen, oxo, nitro, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or R^{a}, together with R and the nitrogen atom to which it is attached, optionally forms a 4-8 membered heterocycle optionally substituted with one or more instances of J^{E}; or R^{b} and R^{c}, together with the nitrogen atom to which they are attached, optionally forms a 4-8 membered heterocycle optionally substituted with one or more instances of J^{E}.

In one specific aspect, each R^{a} independently is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 4-8 membered heterocyclic ring; and each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, form a 4-8 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

In another specific aspect, each R^{a} is independently as described in the preceeding paragraph; and each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, optionally form a 5-7 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

Each R is independently —H or a C₁₋₆ aliphatic group optionally substituted with one or more instances of J^{D}.

Each R′ is independently —H or a C₁₋₆ aliphatic group optionally substituted with one or more instances of J^{D}; or two R′, together with the nitrogen atom to which they are attached, optionally form a 4-8 membered heterocycle optionally substituted with one or more instances of J^{E}.

Each R″ is a C₁₋₆ aliphatic group optionally substituted with one or more instances of J^{D}.

Each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

Each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D}.

Each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}.

n is 0 or 1. In one aspect, n is 0.

A second set of values of the variables of Structural Formula (I) is as follows:

Y is optionally substituted C₃₋₆ cycloalkyl, optionally substituted C₄₋₆ cycloalkenyl, —(C₂ aliphatic group)-R¹, optionally substituted phenyl, or optionally substituted 5-6 membered heteroaryl, and wherein said C₂ aliphatic group is optionally substituted. In one specific aspect, Y is optionally substituted phenyl, optionally substituted thienyl, or optionally substituted pyridyl. In another specific aspect, Y is optionally substituted phenyl. Suitable substituents for the values of Y are as described above in the first set of variables of Structural Formula (I).

The remaining variables of Structural Formula (I) are each and independently as described above in the first set of variables of Structural Formula (I).

A third set of values of the variables of Structural Formula (I) is as follows:

Y is optionally substituted C₃₋₆ cycloalkyl or optionally substituted C₄₋₆ cycloalkenyl. In one specific aspect, Y is optionally substituted C₄₋₆ cycloalkenyl. In another specific aspect, Y is optionally substituted cyclohexenyl. Suitable substituents for the values of Y are as described above in the first set of variables of Structural Formula (I).

The remaining variables of Structural Formula (I) are each and independently as described above in the first set of variables of Structural Formula (I).

A fourth set of values of the variables of Structural Formula (I) is as follows:

Y is as described above in any one of the first, second, and third sets of values of the variables of Structural Formula (I), and each J^{Y} is independently selected from the group consisting of halogen, —CN, nitro, R^{a}, —OR^{a}, —COR^{a}, and —NRR^{a}. In one specific aspect, each J^{Y} is independently selected from the group consisting of halogen, —CN, nitro, C₁₋₆ alkyl, C₁₋₆ haloalkyl, —OH, —O(C₁₋₆ alkyl), —O(phenyl), —O(5-6 membered heteroaryl), —NH₂, —NH(C₁₋₆ alkyl), —N(C₁₋₆ alkyl)₂, and —C(O)(C₁₋₆ alkyl). In another specific aspect, each J^{Y} is independently selected from the group consisting of halogen, —CN, nitro, C₁₋₆ alkyl, C₁₋₆ haloalkyl, —OH, —O(C₁₋₆ alkyl), —NH₂, —NH(C₁₋₆ alkyl), —N(C₁₋₆ alkyl)₂, and —C(O)(C₁₋₆ alkyl). In yet another specific aspect, each J^{Y} is independently selected from the group consisting of halogen, —CN, nitro, methyl, ethyl, —CF₃, —OH, —OMe, —NH₂, and —C(O)Me.

The remaining variables of Structural Formula (I) are each and independently as described above in the first set of variables of Structural Formula (I).

A fifth set of values of the variables of Structural Formula (I) is as follows:

Y is —(C₂ aliphatic group)-R¹, and wherein said C₂ aliphatic group is optionally substituted. In one specific aspect, Y is —CH₂—CH₂—R¹, —CH═CH—R′, or —C≡CR¹. In another specific aspect, Y is —C≡CR¹. Typical examples of substituents suitable for the C₂ aliphatic group of —(C₂ aliphatic group)-R¹ include halogen, —CN, C₁₋₂ alkyl, C₁₋₂ haloalkyl, hydroxy, and methoxy.

The remaining variables of Structural Formula (I) are each and independently as described above in the first set of variables of Structural Formula (I).

A sixth set of values of the variables of Structural Formula (I) is as follows:

Y is as described above in any one of the first through fifth sets of values of the variables of Structural Formula (I).

R¹ is an optionally substituted C₁₋₆ alkyl or optionally substituted C₃₋₈ carbocyclic group. Suitable substituents for the values of R¹ are each and independently as described above in the first set of variables of Structural Formula (I). In one specific aspect, R¹ is an optionally substituted C₁₋₆ alkyl or C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In another specific aspect, R¹ is an optionally substituted C₁₋₆ alkyl. In yet another specific aspect, R¹ is C₁₋₆ alkyl optionally substituted with one or more substituents selected from the group consisting of halogen, —CN, —OH, and —O(C₁-C₆ alkyl).

The remaining variables of Structural Formula (I) are each and independently as described above in the first set of variables of Structural Formula (I).

A seventh set of values of the variables of Structural Formula (I) is as follows:

Y is as described above in any one of the first through fifth sets of values of the variables of Structural Formula (I).

R¹ is as described above in the sixth set of values of the variables of Structural Formula (I).

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group. Suitable substituents for the values of R² are each and independently as described above in the first set of variables of Structural Formula (I). In one specific aspect, R² is C₅-C₈ cycloalkyl optionally substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In another specific aspect, R² is optionally substituted cyclohexyl. In yet another specific aspect, R² is cyclohexyl optionally substituted with one or more instances of J^{2B} independently selected from the group consisting of halogen, —CN, —OH, —NH₂, —NH(C₁₋₆ alkyl), —N(C₁₋₆ alkyl)₂, —O(C₁₋₆ alkyl), and C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl).

The remaining variables of Structural Formula (I) are each and independently as described above in the first set of variables of Structural Formula (I).

An eighth set of values of the variables of Structural Formula (I) is as follows:

X is [—C(O)—C(R⁴R⁵)—N(R)—]ₙ—C(O)C(R⁶R⁷R⁸) or —P(O)(OR³)₂.

R¹ and R² are each and independently as described above in the seventh set of values of the variables of Structural Formula (I).

The remaining variables of Structural Formula (I) are each and independently as described above in the first set of variables of Structural Formula (I).

A ninth set of values of the variables of Structural Formula (I) is as follows:

X, Y, R¹ and R² are each and independently as described above in any one of the first through eighth set of values of the variables of Structural Formula (I).

Each Q independently is selected from the group consisting of halogen; cyano; nitro; —OR^{a}; —SR^{a}, —S(O)R^{a}; —SO₂R^{a}; —NRR^{a}, —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Suitable substituents for the values of Q are each and independently as described above in the first set of variables of Structural Formula (I).

The remaining variables of Structural Formula (I) are each and independently as described above in the first set of variables of Structural Formula (I).

A tenth set of values of the variables of Structural Formula (I) is as follows:

X, Y, Q, R¹ and R² are each and independently as described above in any one of the first through ninth set of values of the variables of Structural Formula (I).

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 4-8 membered heterocyclic ring. Suitable substituents for the values of R^{a} are each and independently as described above in the first set of variables of Structural Formula (I).

Each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, form a 4-8 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

The remaining variables of Structural Formula (I) are each and independently as described above in the first set of variables of Structural Formula (I).

An eleventh set of values of the variables of Structural Formula (I) is as follows:

X, Y, Q, R¹, R², R^{a}, R^{b} and R^{c} are each and independently as described above in any one of the first through ninth set of values of the variables of Structural Formula (I).

Each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. Suitable substituents for the values of R³ are each and independently as described above in the first set of variables of Structural Formula (I).

Each of R⁴, R⁵, R⁶, and R⁷ independently is —H; or C₁₋₆ alkyl optionally substituted with one or more substitutents selected from the group consisting of —OH, —NH₂, —NHC(═NH)NH₂, —CO₂H, —C(O)NH₂, phenyl, hydroxyphenyl, imidazole, and indole.

R⁸ independently is —H, halogen, cyano, —OR^{b}, —NR^{b}R^{c} optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. Suitable substituents for the values of R⁸ are each and independently as described above in the first set of variables of Structural Formula (I).

The remaining variables of Structural Formula (I) are each and independently as described above in the first set of variables of Structural Formula (I).

A twelfth set of values of the variables of Structural Formula (I) is as follows:

X, Y, Q, R¹, R², R^{a}, R^{b} and R^{c} are each and independently as described above in any one of the first through ninth set of values of the variables of Structural Formula (I).

Each R³ independently is —H or C₁₋₆ alkyl optionally substituted with one or more instances of J^{D}.

R⁴ and R⁶ are each independently —H or C₁₋₆ alkyl.

R⁵ and R⁷ are each independently —H or C₁₋₆ alkyl optionally substituted with one or more instances of J^{D}.

The remaining variables of Structural Formula (I) are each and independently as described above in the first set of variables of Structural Formula (I).

A thirteenth set of values of the variables of Structural Formula (I) is as follows:

X, Y, Q, R¹, R², R³, R⁴, R⁵, R⁶, R⁷, R^{a}, R^{b} and R^{c} are each and independently as described above in any one of the first through twelfth set of values of the variables of Structural Formula (I).

R⁸ is —NR^{b}R^{c}.

The remaining variables of Structural Formula (I) are each and independently as described above in the first set of variables of Structural Formula (I).

A fourteenth set of values of the variables of Structural Formula (I) is as follows:

X, Y, Q, R¹, R², R³, R⁴, R⁵, R⁶, R⁷, R⁸, R^{a}, R^{b} and R^{c} are each and independently as described above in any one of the first through thirteenth set of values of the variables of Structural Formula (I).

Each of J^{1A} and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl.

Each of J^{1B} and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Each of J^{1C} and J^{9C} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

The remaining variables of Structural Formula (I) are each and independently as described above in the first set of variables of Structural Formula (I).

A fifteenth set of values of the variables of Structural Formula (I) is as follows:

X, Y, Q, R¹, R², R³, R⁸, R^{a}, R^{b}, R^{c}, J^{1A}, J^{9A}, J^{1B}, J^{9B}, J^{1C}, and J⁹ are each and independently as described above in any one of the first through fourteenth set of values of the variables of Structural Formula (I).

Each of R⁴ and R⁶ is independently —H or C₁₋₆ alkyl.

Each of R⁵ and R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, optionally form a 5-7 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

The remaining variables of Structural Formula (I) are each and independently as described above in the first set of variables of Structural Formula (I).

A sixteenth set of values of the variables of Structural Formula (I) is as follows:

X, Y, Q, R¹, R², R⁴, R⁵, R⁶, R⁷, R⁸, R^{a}, R^{b}, R^{c}, J^{1A}, J^{9A}, J^{1B}, J^{9B}, J^{1C}, and J^{9C} are each and independently as described above in any one of the first through fifteenth set of values of the variables of Structural Formula (I).

R³ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In one specific aspect, R³ is —H or C₁₋₆ alkyl optionally substituted with one or more substituents selected from the group consisting of halogen, —CN, —OH, —NH₂, —NH(C₁-C₆ alkyl), —N(C₁-C₆ alkyl)₂, —O(C₁-C₆ alkyl), —O(C₁-C₆ haloalkyl), C₃₋₇ cycloalkyl, C₃₋₇ cyclo(haloalkyl), and phenyl. In another specific aspect, R³ is —H or C₁₋₆ alkyl optionally substituted with one or more substituents selected from the group consisting of halogen, —CN, —OH, —O(C₁-C₆ alkyl), C₃₋₇ cycloalkyl, and phenyl.

The remaining variables of Structural Formula (I) are each and independently as described above in the first set of variables of Structural Formula (I).

A seventeenth set of values of the variables of Structural Formula (I) is as follows:

X, Y, Q, R¹, R², R³, R⁵, R⁷, R⁸, R^{a}, R^{b}, R^{c}, J^{1A}, J^{9A}, J^{1B}, J^{9B}, J^{1C}, and J^{9C} are each and independently as described above in any one of the first through sixteenth set of values of the variables of Structural Formula (I).

Each of R⁴ and R⁶ is independently —H.

The remaining variables of Structural Formula (I) are each and independently as described above in the first set of variables of Structural Formula (I).

An eighteenth set of values of the variables of Structural Formula (I) is as follows:

X, Y, Q, R¹, R², R³, R⁴, R⁵, R⁶, R⁷, R⁸, R^{a}, J^{1A}, J^{9A}, J^{1B}, J^{9B}, J^{1C}, and J^{9C} are each and independently as described above in any one of the first through seventeenth set of values of the variables of Structural Formula (I).

Each of R^{b} and R^{c} independently is —H or C₁₋₆ alkyl, or optionally, together with the nitrogen atom to which they are attached, form an optionally substituted, 5-7 membered, heterocyclic ring.

The remaining variables of Structural Formula (I) are each and independently as described above in the first set of variables of Structural Formula (I).

A nineteenth set of values of the variables of Structural Formula (I) is as follows:

X, Y, Q, R¹, R², R³, R⁴, R⁵, R⁶, R⁷, R⁸, R^{a}, R^{b}, R^{c}, J^{1A}, J^{9A}, J^{1B}, J^{9B}, J^{1C}, and J^{9C} are each and independently as described above in any one of the first through seventeenth set of values of the variables of Structural Formula (I).

Ring B is optionally substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

Ring C is optionally further substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

The remaining variables of Structural Formula (I) are each and independently as described above in the first set of variables of Structural Formula (I).

A twentieth set of values of the variables of Structural Formula (I) is as follows:

X, Y, Q, R¹, R², R³, R⁴, R⁵, R⁶, R⁷, R⁸, R^{a}, R^{b}, R^{c}, J^{1A}, J^{9A}, J^{1B}, J^{9B}, J^{1C}, and J^{9C} are each and independently as described above in any one of the first through seventeenth set of values of the variables of Structural Formula (I).

Ring B is C₃₋₈ cycloalkyl optionally substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

The remaining variables of Structural Formula (I) are each and independently as described above in the first set of values of the variables of Structural Formula (I).

In another embodiment, the compounds of the invention are represented by Structural Formula (II):

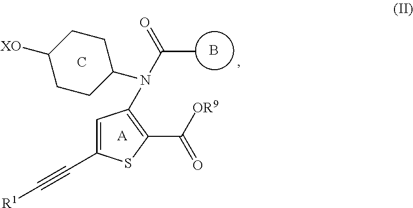

or pharmaceutically acceptable salts thereof, wherein each of Rings A, B and C is independently and optionally substituted. The variables of Structural Formula (II) are each and independently as described above in any one of the first through twentieth sets of values of the variables of Structural Formula (I). Suitable variables for Rings A, B and C are each and independently as described above in the first set of values of the variables of Structural Formula (I).

In a twenty first set of values of the variables of Structural Formula (II):

R¹ is an optionally substituted C₁₋₆ alkyl or optionally substituted C₃₋₈ carbocyclic group.

R² is an optionally substituted C₁₋₆ aliphatic, optionally substituted C₃₋₈ carbocyclic, optionally substituted 4-8 membered heterocyclic, optionally substituted phenyl, or optionally substituted 5-6 membered heteroaryl group.

The remaining variables of Structural Formula (I) are each and independently as described above in the first set of values of the variables of Structural Formula (I).

In a twenty second set of values of the variables of Structural Formula (II):

R¹ and R² are each independently as described above in the twenty first set of values of the variables of Structural Formula (II).

X is [—C(O)—C(R⁴R⁵)—N(R)—]ₙ—C(O)C(R⁶R⁷R⁸) or —P(O)(OR³)₂.

The remaining variables of Structural Formula (I) are each and independently as described above in the first set of values of the variables of Structural Formula (I).

In a twenty third set of values of the variables of Structural Formula (II):

R¹, R² and X are each independently as described above in the twenty second set of values of the variables of Structural Formula (II).

Q independently is selected from the group consisting of halogen; cyano; nitro; —OR^{a}; —SR^{a}; —S(O)R^{a}; —SO₂R^{a}; —NRR^{a}; —C(O)R^{a}; —C(O)OR^{a}; —OC(O)R^{a}; —OC(O)OR^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Alternatively, Q independently is selected from the group consisting of halogen; cyano; nitro; —OR^{a}; —SR^{a}; —S(O)R^{a}; —SO₂R^{a}; —NRR^{a}, —C(O)R^{a}; —C(O)OR^{a}; —OC(O)R^{a}; —NRC(O)R^{a}; —C(O)NRR^{a}; —NRC(O)NRR^{a}; —NRC(O)OR^{a}; —NRC(═NR)NRR^{a}, —OCONRR^{a}; —C(O)NRC(O)OR^{a}; —C(═NR)R^{a}; —C(═NOR)R^{a}; —SO₂NRR^{a}; —NRSO₂R^{a}; —NRSO₂NRR^{a}; —OP(O)(OR^{a})OR^{a}; optionally substituted C₃₋₈ carbocyclic; 4-8 membered, optionally substituted heterocyclyl; optionally substituted phenyl; and optionally substituted, 5-6 membered heteroaryl. Suitable substitutents for the C₃₋₈ carbocyclic, heterocyclyl, phenyl; and heteroaryl groups are each and independently as described above in the first set of values of the variables of Structural Formula (I).

The remaining variables of Structural Formula (I) are each and independently as described above in the first set of values of the variables of Structural Formula (I).

In a twenty fourth set of values of the variables of Structural Formula (II):

R¹, R², X, and Q are each independently as described above in the twenty third set of values of the variables of Structural Formula (II).

R^{a} is —H, optionally substituted C₁₋₆ aliphatic, optionally substituted C₃₋₆ carbocyclic, optionally substituted 4-8 membered heterocyclic, optionally substituted phenyl, or optionally substituted 5-6 remembered heteroaryl; or optionally R^{a}, together with R and the nitrogen atom to which it is attached, forms an optionally substituted 4-8 membered heterocyclic ring. Suitable substitutents for the aliphatic, carbocyclic, heterocyclyl, phenyl; and heteroaryl groups are each and independently as described above in the first set of values of the variables of Structural Formula (I).

Each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, form a 4-8 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

The remaining variables of Structural Formula (I) are each and independently as described above in the first set of values of the variables of Structural Formula (I).

In a twenty fifth set of values of the variables of Structural Formula (II):

R¹, R², X, Q, R^{a}, R^{b} and R^{c} are each independently as described above in the twenty fourth set of values of the variables of Structural Formula (II).

R³ is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl.

Each of R⁴, R⁵, R⁶, and R⁷ independently is —H; or C₁₋₆ alkyl optionally substituted with one or more substitutents selected from the group consisting of —OH, —NH₂, —NHC(═NH)NH₂, —CO₂H, —C(O)NH₂, phenyl, hydroxyphenyl, imidazole, and indole.

R⁸ independently is —H, halogen, cyano, —OR^{b}, —NR^{b}R^{c}, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl. Suitable substituents for the carbocyclic, heterocyclic, phenyl, and heteroaryl groups are each and independently as described above in the first set of values of the variables of Structural Formula (I).

The remaining variables of Structural Formula (II) are each and independently as described above in the first set of values of the variables of Structural Formula (I).

In a twenty sixth set of values of the variables of Structural Formula (II):

R¹, R², X, Q, R^{a}, R^{b} and R^{c} are each independently as described above in the twenty fifth set of values of the variables of Structural Formula (II).

R³ is —H or optionally substituted C₁₋₆ alkyl.

R⁴ and R⁶ are each independently —H or C₁₋₆ alkyl.

R⁵ and R⁷ are each independently —H or optionally substituted C₁₋₆ alkyl.

The remaining variables of Structural Formula (II) are each and independently as described above in the first set of values of the variables of Structural Formula (I).

In a twenty seventh set of values of the variables of Structural Formula (II):

R¹, R², R³, R⁴, R⁵, R⁶, R⁷, X, Q, R^{a}, R^{b} and R^{c} are each independently as described above in the twenty sixth set of values of the variables of Structural Formula (II).

R⁸ is —NR^{b}R^{c}.

The remaining variables of Structural Formula (II) are each and independently as described above in the first set of values of the variables of Structural Formula (I).

In a twenty eighth set of values of the variables of Structural Formula (II):

R¹, R², R³, R⁴, R⁵, R⁶, R⁷, R⁸, X, Q, R^{a}, R^{b} and R^{c} are each independently as described above in the twenty seventh set of values of the variables of Structural Formula (II).

Each of J^{1A} and J^{9A} independently is halogen, oxo, —CN, —OR^{a}, —NRR^{a}, —OCOR^{a}, —OCOOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), optionally substituted 5-6 membered heterocyclyl, or optionally substituted phenyl. Alternatively, each of J^{1A} and J^{9A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl. Alternatively, each of J^{1A} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), or phenyl; and each of J^{9A} independently is halogen, oxo, —CN, —OR^{a}, —NRR^{a}, —OCOR^{a}, —OCOOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), phenyl, or 5-6 membered heterocyclyl optionally substituted with one or more substitutents selected from the group consisting of oxo and C₁₋₆ alkyl.

Each of J^{1B} and J^{9B} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

Each of J^{1C} and J^{9C} independently is halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, or a C₁-C₆ aliphatic group optionally substituted with one or more substituents selected from the group consisting of halogen, oxo, —CN, —OR^{a}, —NRR^{a}, —OCOR^{a}, —COR^{a}, —CO₂R^{a}, —NRC(O)R^{a}, —C(O)NRR^{a}, —NRC(O)NRR^{a}, —NRC(O)OR^{a}, —OCONRR^{a}, C₃₋₈ cycloalkyl, C₃₋₈ cyclo(haloalkyl), and phenyl.

The remaining variables of Structural Formula (II) are each and independently as described above in the first set of values of the variables of Structural Formula (I).

In a twenty ninth set of values of the variables of Structural Formula (II):

R¹, R², R³, R⁸, X, Q, R^{a}, J^{1A}, J^{9A}, J^{1B}, J^{9B}, J^{1C}, and J^{9C} are each independently as described above in the twenty eighth set of values of the variables of Structural Formula (II).

Each of R⁴ and R⁶ is independently —H or C₁₋₆ alkyl.

Each of R⁵ and R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, optionally form a 5-7 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

The remaining variables of Structural Formula (II) are each and independently as described above in the first set of values of the variables of Structural Formula (I).

In a thirtieth set of values of the variables of Structural Formula (II):

R¹, R², R⁴, R⁵, R⁶, R⁷, R⁸, X, Q, R^{a}, R^{b}, R^{c}, J^{1A}, J^{9A}, J^{1B}, J^{9B}, J^{1C} and J^{9C} are each independently as described above in the twenty ninth set of values of the variables of Structural Formula (II).

R³ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. Specifically, R³ is —H or C₁₋₆ alkyl optionally substituted with one or more substituents selected from the group consisting of halogen, —CN, —OH, —NH₂, —NH(C₁-C₆ alkyl), —N(C₁-C₆ alkyl)₂, —O(C₁-C₆ alkyl), —O(C₁-C₆ haloalkyl), C₃₋₇ cycloalkyl, C₃₋₇ cyclo(haloalkyl), and phenyl. Specifically, R³ is —H or C₁₋₆ alkyl optionally substituted with one or more substituents selected from the group consisting of halogen, —CN, —OH, —O(C₁-C₆ alkyl), C₃₋₇ cycloalkyl, and phenyl.

The remaining variables of Structural Formula (II) are each and independently as described above in the first set of values of the variables of Structural Formula (I).

In a thirty first set of values of the variables of Structural Formula (II):

R¹, R², R³, R⁵, R⁷, R⁸, X, Q, R^{a}, R^{b}, R^{c}, J^{1A}, J^{9A}, J^{1B}, J^{9B}, J^{1C} and J^{9C} are each independently as described above in the thirtieth set of values of the variables of Structural Formula (II).

Each of R⁴ and R⁶ is independently —H.

The remaining variables of Structural Formula (II) are each and independently as described above in the first set of values of the variables of Structural Formula (I).

In a thirty second set of values of the variables of Structural Formula (II):

R¹, R², R³, R⁵, R⁷, R⁸, X, Q, R^{a}, J^{1A}, J^{9A}, J^{1B}, J^{9B}, J^{1C} and J^{9C} are each independently as described above in the thirty first set of values of the variables of Structural Formula (II).

Each of R^{b} and R^{c} independently is —H or C₁₋₆ alkyl, or optionally, together with the nitrogen atom to which they are attached, form an optionally substituted, 5-7 membered, heterocyclic ring.

The remaining variables of Structural Formula (II) are each and independently as described above in the first set of values of the variables of Structural Formula (I).

In a thirty third set of values of the variables of Structural Formula (II):

R¹, R², R³, R⁵, R⁷, R⁸, X, Q, R^{a}, R^{b}, R^{c}, J^{1A}, J^{9A}, J^{1B}, J^{9B}, J^{1C} and J^{9C} are each independently as described above in the thirty second set of values of the variables of Structural Formula (II).

Ring B is optionally substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

Ring C is optionally further substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

The remaining variables of Structural Formula (II) are each and independently as described above in the first set of values of the variables of Structural Formula (I).

In a thirty fourth set of values of the variables of Structural Formula (II):

R¹, R², R³, R⁵, R⁷, R⁸, X, Q, R^{a}, R^{b}, R^{c}, J^{1A}, J^{9A}, J^{1B}, J^{9B}, J^{1C} and J^{9C} are each independently as described above in the thirty second set of values of the variables of Structural Formula (II).

Ring B is optionally substituted C₃₋₈ cycloalkyl optionally substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

Ring C is optionally further substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

The remaining variables of Structural Formula (II) are each and independently as described above in the first set of values of the variables of Structural Formula (I).

In a thirty fifth set of values of the variables of Structural Formula (II):

Ring A is not further substituted, and the remaining variables of Structural Formula (II) are each independently as described above in any one of the first through thirty fourth sets of values of the variables of Structural Formula (II).

In yet another embodiment, the compounds of the invention are represented by any one of Structural Formulae (III)-(VII) or pharmaceutically acceptable salts thereof:

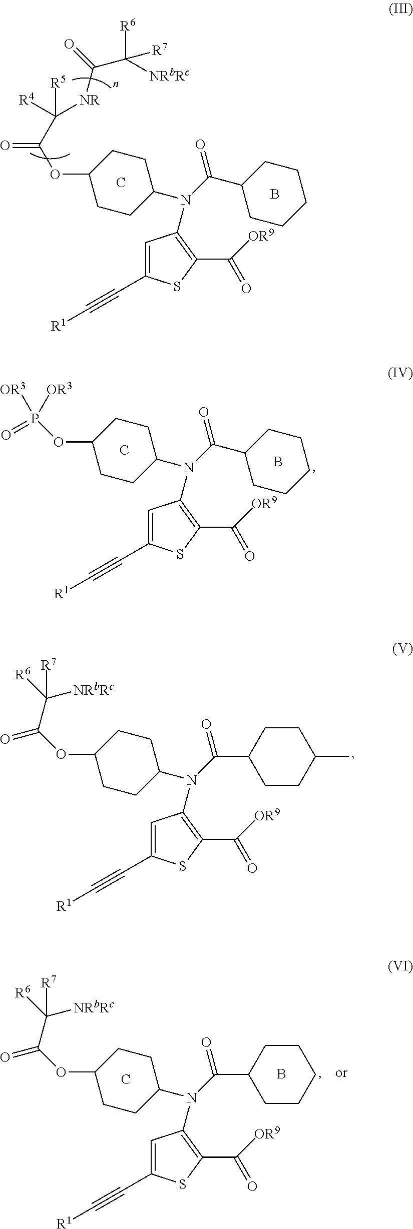

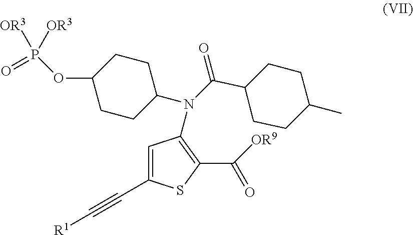

wherein each ring B independently is optionally substituted cyclohexyl, and each ring C is optionally substituted cyclohexyl. The variables of these structural formulae are each and independently as described above in any one of the first through thrifty fifth sets of values of the variables of Structural Formula (II). Suitable variables for Rings B and C are each and independently as described above in the first set of values of the variables of Structural Formula (I).

In a thirty sixth set of values of the variables of Structural Formulae (III)-(VII):

Each R⁶ independently is —H.

Each R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl.

The remaining variables of Structural Formulae (III)-(VII) are each and independently as described above in the first set of values of the variables of Structural Formula (I).

In a thirty seventh set of values of the variables of Structural Formulae (III)-(VII):

Each R³ independently is —H or C₁₋₆ alkyl.

Each R⁶ independently is —H.

Each R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl.

The remaining variables of Structural Formulae (III)-(VII) are each and independently as described above in the first set of values of the variables of Structural Formula (I).

In a thirty eighth set of values of the variables of Structural Formulae (III)-(VII):

Each R¹ independently is an optionally substituted C₁₋₆ alkyl or optionally substituted C₃₋₈ carbocyclic group. Suitable substituents are as described above in the first set of values of the variables of Structural Formula (I).

Each R³ independently is —H or C₁₋₆ alkyl.

Each R⁶ independently is —H.

Each R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl.

The remaining variables of Structural Formulae (III)-(VII) are each and independently as described above in the first set of values of the variables of Structural Formula (I).

In a thirty ninth set of values of the variables of Structural Formulae (III)-(VII):

Each R¹ is optionally substituted C₁₋₆ alkyl or optionally substituted C₃₋₈ cycloalkyl, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. In one aspect, R¹ is C₁₋₆ alkyl or C₃₋₈ cycloalkyl, each of which optionally and independently substituted with one or more substituents selected from the group consisting of halogen, —CN, —OH, —O(C₁₋₆ alkyl), and —O(C₁₋₆ haloalkyl). In another aspect, R¹ is C₁₋₆ alkyl or C₃₋₈ cycloalkyl. In yet another aspect, R¹ is C₁₋₆ alkyl. In yet another aspect, R¹ is t-butyl or isopropyl.

Each R³ independently is —H or C₁₋₆ alkyl.

Each R⁶ independently is —H.

Each R⁷ independently is —H or C₁₋₆ alkyl optionally substituted with one or more substitutents selected from the group consisting of —OH, —NH₂, —NH—C(═NH)—NH₂, —CO₂H, and —C(O)NH₂.

Each of R^{b} and R^{c} independently is —H or C₁₋₆ alkyl.

The remaining variables of Structural Formulae (III)-(VII) are each and independently as described above in the first set of values of the variables of Structural Formula (I).

In a fortieth set of values of the variables of Structural Formulae (III)-(VII):

R¹, R³, R⁶, R⁷, R^{b}, and R^{c} are each independently as described above in any one of the first through thirty ninth set of values of the variables of Structural Formulae (III)-(VII).

Each R⁹ independently is —H, or an optionally substituted C₁₋₆ aliphatic or optionally substituted carbocyclic group. Suitable substituents for the C₁₋₆ aliphatic and carbocyclic groups are each and independently as described above in the first set of values of the variables of Structural Formula (I).

The remaining variables of Structural Formulae (III)-(VII) are each and independently as described above in the first set of values of the variables of Structural Formula (I).

In a forty first set of values of the variables of Structural Formulae (III)-(VII):

R¹, R³, R⁶, R⁷, R^{b}, and R^{c} are each independently as described above in any one of the first through thirty ninth set of values of the variables of Structural Formulae (III)-(VII).

Each R⁹ independently is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —OCOO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), phenyl, and 5-6 membered heterocycle optionally substituted with one or more substitutents selected from oxo and C₁-C₆ alkyl. Alternatively, each R⁹ independently is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl.

The remaining variables of Structural Formulae (III)-(VII) are each and independently as described above in the first set of values of the variables of Structural Formula (I).

In a forty second set of values of the variables of Structural Formulae (III)-(VII):

R¹, R³, R⁶, R⁷, R^{b}, and R^{c} are each independently as described above in any one of the first through thirty ninth set of values of the variables of Structural Formulae (III)-(VII).

Each R⁹ is —H.

The remaining variables of Structural Formulae (III)-(VII) are each and independently as described above in the first set of values of the variables of Structural Formula (I).

In some embodiments, the compounds of the invention are represented by

Structural Formula (I) or (II), wherein:

R² is C₅-C₈ cycloalkenyl optionally substituted with one or more substituents selected from the group consisting of halogen; oxo; —CN; —OH; —NH₂; —NH(C₁-C₆ alkyl); —N(C₁-C₆ alkyl)₂; —OCO(C₁-C₆ alkyl); —CO(C₁-C₆ alkyl); —CO₂H; —CO₂(C₁-C₆ alkyl); —O(C₁-C₆ alkyl); —O(C₁-C₆ haloalkyl); and a C₁-C₆ aliphatic group optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl. Specifically, R² is optionally substituted cyclohexenyl. More specifically, R² is cyclohexenyl optionally substituted with one or more instances of J^{2B} independently selected from the group consisting of halogen, —CN, —OH, —NH₂, —NH(C₁₋₆ alkyl), —N(C₁₋₆ alkyl)₂, —O(C₁₋₆ alkyl), and C₁₋₆ alkyl optionally substituted with one or more substituents selected from the group consisting of halogen, —CN, —OH, —NH₂, —NH(C₁₋₆ alkyl), —N(C₁₋₆ alkyl)₂, —O(C₁₋₆ alkyl), and —O(C₁₋₆ haloalkyl).

The remaining variables of Structural Formulae (I)-(II) are each and independently as described above in any one of the sets of values of the variables of Structural Formula (I).

In some embodiments, the compounds of the invention are represented by any one of the structural formulae depicted in FIG. 1, or pharmaceutically acceptable salts thereof. In other embodiments, the compounds of the invention are represented by any one of the structural formulae depicted in FIG. 2 or pharmaceutically acceptable salts thereof.

As used herein, a reference to compound(s) of the invention, for example compound(s) of Structural Formula (I), or compound(s) of claim 1, will include pharmaceutically acceptable salts thereof.

The compounds according to the invention described herein can be prepared by any suitable method known in the art. For example, the compounds can be prepared in accordance with procedures described in U.S. Pat. No. 6,881,741, US 2005/0009804, US 2006/0276533, WO 2002/100851, and WO 08/58393, the disclosures of which are hereby incorporated by reference.

In one embodiment, the compounds of the invention can be prepared as depicted in General Schemes 1 and 2. For example, the compounds of Structural Formulae (I), (II), (III), (IV), (V), (VI), and (VII) can be prepared as shown in General Schemes 1-2. Any suitable condition known in the art can be employed for each step described in the schemes. Specific exemplary conditions are described in the schemes, and exemplary detailed procedures are described below in the Exemplification section.

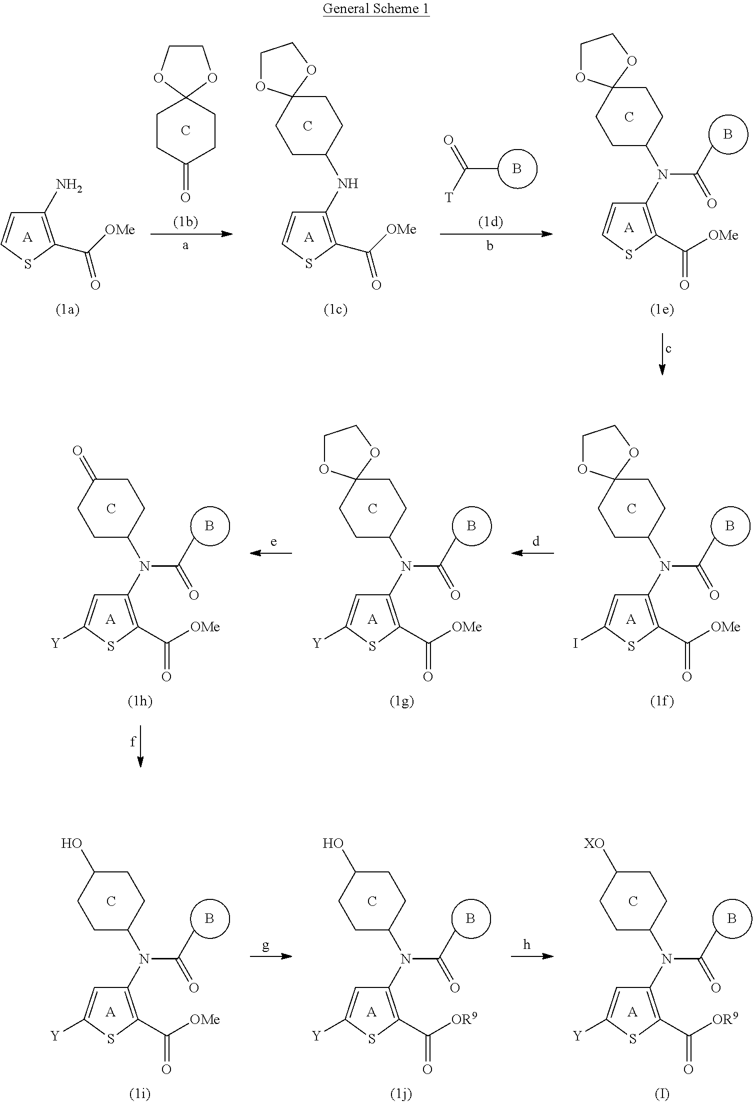

Representative conditions: (a) NaBH₄, AcOH; (b) i. For T=OH, SOCl₂, DCM, cat. DMF, or for T=Cl, ii. Pyridine, DCE, DCM; (c) n-BuLi, THF, iPr₂NH, I₂; (d) For Y═(C₂ aliphatic group)-R²: YH, CuI, Pd(PPh₃)₂Cl₂, THF, Et₃N; for Y=carbocycle, heterocycle, aryl, or heoayl: YB(OR^{k})₂, Pd(OAc)₂, K₃PO₄, toluene, heat; (e) HCl, H₂O; (f) NaBH₄, THF, H₂O; (g) For R⁹═H, THF, H₂O, LiOH; (h) For X═[C(O)C(R⁴R⁵)N(R)—]ₙ—C(O)C(R⁶R⁷)NR^{b}R^{c}: EDC, HO—[C(O)C(R⁴R⁵)N(R)—]ₙ—C(O)C(R⁶R⁷)NR^{b}R^{c}, EDC, Et₃N, DMAP, CH₂Cl₂; for X=—P(O)(OR³)₂: i. R₂N—P(OR³)₂, CH₂Cl₂, tetrazole, PhNCO, H₂O₂, ii. for R³═H, H₂, Pd; For X═C(O)R²: HOC(O)R², SOCl₂, toluene

As shown in General Scheme 1, the compounds of Structural Formula (I) can be prepared from compound (1j) by suitable reaction with X—OH for X=—[C(O)C(R⁴R⁵)N(R)]ₙC(O)C(R⁶R⁷)NR^{b}R^{c} or X=—(CO)R², or with (R^{k})₂N—P(OR³)₂ (wherein R^{k} is typically —H, C₁₋₆ alkyl (e.g., methy, ethyl), benzyl, etc.) for X=—P(O)(OR³)₂. The compounds described in General Scheme 1, including compounds (1c), (1e), (1f), (1g), (1 h), (1i), and (1j), can generally be prepared by any suitable method known in the art. In a specific embodiment, the methods further comprise the step of preparing compound (1j). In another specific embodiment, the methods further comprise the step of preparing compound (1j) as described in General Scheme 1. Specifically, compound (1j) can be prepared starting from compound (1f). Reaction of compound (1f) with YH for Y═(C₂ aliphatic group)-R¹ (such as —CH₂CH₂R′, —CH═CHR¹, or —C≡CR¹) or with YB(OR^{k})₂ (where R^{k} is typically —H, C₁₋₆ alkyl or benzyl) for Y=carbocycle, heterocycle, aryl, or heteroaryl can produce compound (1g). Subsequent treatment of compound (1g) with an acid (e.g., HCl) in an aqueous condition can produce compound (1 h). Reduction of the ketone group at ring C of compound (1 h) can produce compound (1i). Treatment of compound (1i) under a suitable hydrolysis condition, for example in the presence of LiOH in H₂O, can produce compound (1j) where R⁹ is —H. If desired, compound (1j) can be further reacted with a suitable reagent(s) known in the art to form compounds having other than —H for R⁹.

In another embodiment, the present invention provides methods of preparing a compound represented by Structural Formula (II). General Scheme 2 shows a general synthetic scheme for the compounds of Structural Formula (II). The synthetic details are each and independently as described above for General Scheme 1. For example, compounds (1f), (2g), (2h), (2i), and (2j) are each and independently as described in General Scheme 1 for compounds (1f), (1g), (1h), (1i), and (1j), respectively. The methods comprise the step of reacting compound (2j) with X—OH for X=—[C(O)C(R⁴R⁵)N(R)]ₙC(O)C(R⁶R⁷)NR^{b}R^{c} and X=—(CO)R², or with (R^{k})₂N—P(OR³)₂ (where R^{k} is typically —H, C₁₋₆ alkyl (e.g., methyl, ethyl), benzyl, etc.) for X=—P(O)(OR³)₂. In a specific embodiment, the methods further comprise the step of preparing compound (2j) as described in the preceding paragraph for compound (1j). Specifically, compound (2j) can be prepared starting from compound (10. Reaction of compound (1f) with R¹—C≡CH can produce compound (2g); subsequent treatment of compound (2g) with an acid (e.g., HCl) in an aqueous condition can produce compound (2h); reduction of the ketone group at ring C of compound (2h) can produce compound (2i); treatment of compound (2i) under a suitable hydrolysis condition, for example in the presence of LiOH in H₂O, can produce compound (2j) where R⁹ is —H. As desired, compound (2j) can be further reacted with a suitable reagent(s) known in the art to form compounds having other than —H for R⁹.

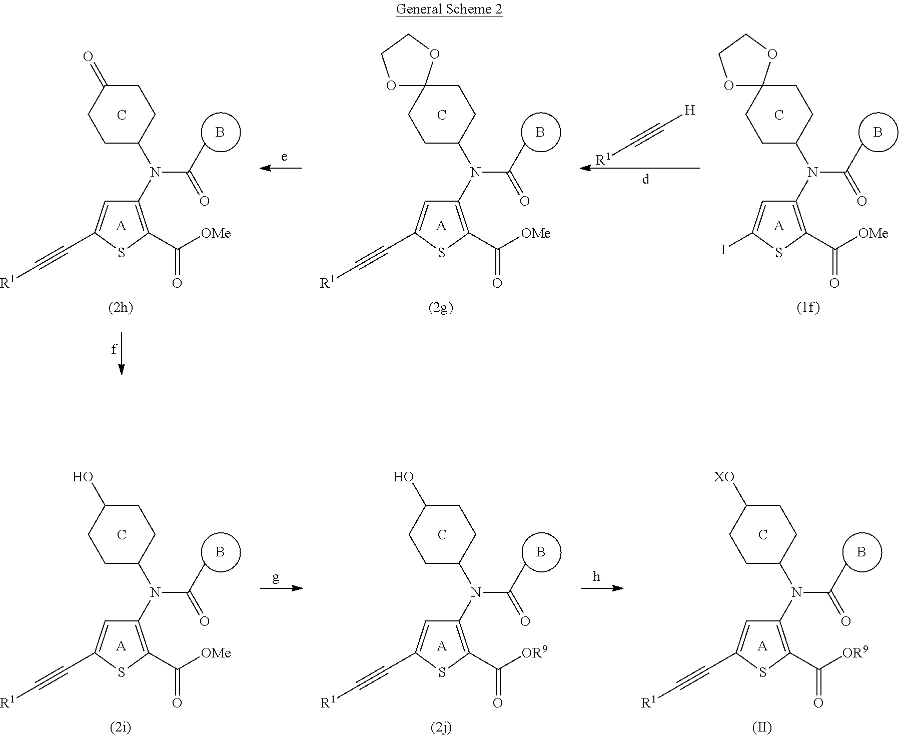

Representative conditions: (d) CuI, Pd(PPh₃)₂Cl₂, THF, Et₃N; (e) HCl, H₂O; (f) NaBH₄, THF, H₂O; (g) For R⁹═H, THF, H₂O, LiOH; (h) For X=[C(O)C(R⁴R⁵)N(R)-]ₙ—C(O)C(R⁶R⁷)NR^{b}R^{c}: EDC, HO—[C(O)C(R⁴R⁵)N(R)—]ₙ—C(O)C(R⁶R⁷)NR^{b}R^{c}, EDC, Et₃N, DMAP, CH₂Cl₂; for X=—P(O)(OR³)₂: i. R₂N—P(OR³)₂, CH₂Cl₂, tetrazole, PhNCO, H₂O₂, ii. for R³═H, H₂, Pd; For X═C(O)R²: HOC(O)R², SOCl₂, toluene

It will be appreciated by those skilled in the art that in the processes of the present invention certain functional groups such as hydroxyl or amino groups in the starting reagents or intermediate compounds may need to be protected by protecting groups. Thus, the preparation of the compounds described above may involve, at various stages, the addition and removal of one or more protecting groups. The protection and deprotection of functional groups is described in “Protective Groups in Organic Chemistry.” edited by J. W. F. McOmie, Plenum Press (1973) and “Protective Groups in Organic Synthesis,” 3rd edition, T. W. Greene and P. G. M. Wuts, Wiley Interscience, and “Protecting Groups,” 3rd edition, P. J. Kocienski, Thieme (2005)

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as illustrated generally below, or as exemplified by particular classes, subclasses, and species of the compounds described above. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. For example, if X is optionally substituted C₁-C₃alkyl or phenyl; X may be either optionally substituted C₁-C₃ alkyl or optionally substituted phenyl. Likewise, if the term “optionally substituted” follows a list, said term also refers to all of the substitutable groups in the prior list unless otherwise indicated. For example: if X is C₁-C₃alkyl or phenyl wherein X is optionally and independently substituted by J^{X}, then both C₁-C₃alkyl and phenyl may be optionally substituted by J^{X}. As is apparent to one having ordinary skill in the art, groups such as H, halogen, NO₂, CN, NH₂, OH, or OCF₃ would not be substitutable groups.

The phrase “up to”, as used herein, refers to zero or any integer number that is equal or less than the number following the phrase. For example, “up to 3” means any one of 0, 1, 2, and 3. As described herein, a specified number range of atoms includes any integer therein. For example, a group having from 1-4 atoms could have 1, 2, 3, or 4 atoms.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “aliphatic” or “aliphatic group”, as used herein, means a straight-chain (i.e., unbranched), or branched, hydrocarbon chain that is completely saturated or that contains one or more units of unsaturation but is non-aromatic. Unless otherwise specified, aliphatic groups contain 1-10 aliphatic carbon atoms. In some embodiments, aliphatic groups contain 1-6 aliphatic carbon atoms. In other embodiments, aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl and acetylene.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds. Each of the “alkyl”, “alkenyl” or “alkynyl” as used herein can be optionally substituted as set forth below. In some embodiments, the “alkyl” is C₁-C₆ alkyl or C₁-C₄ alkyl. In some embodiments, the “alkenyl” is C₂-C₆ alkenyl or C₂-C₄ alkenyl. In some embodiments, the “alkynyl” is C₂-C₆ alkynyl or C₂-C₄ alkynyl.

The term “cycloaliphatic” (or “carbocycle” or “carbocyclyl” or “carbocyclic”) refers to a non-aromatic carbon only containing ring system which can be saturated or contains one or more units of unsaturation, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged carbocyclic ring systems. The term also includes polycyclic ring systems in which the carbocyclic ring can be “fused” to one or more non-aromatic carbocyclic or heterocyclic rings or one or more aromatic rings or combination thereof, wherein the radical or point of attachment is on the carbocyclic ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl,” or “heterocyclic” or “non-aromatic heterocycle”) as used herein refers to a non-aromatic ring system which can be saturated or contain one or more units of unsaturation, having three to fourteen ring atoms in which one or more ring carbons is replaced by a heteroatom such as, N, S, or O. In some embodiments, non-aromatic heterocyclic rings comprise up to three heteroatoms selected from N, S and O within the ring. In other embodiments, non-aromatic heterocyclic rings comprise up to two heteroatoms selected from N, S and O within the ring system. In yet other embodiments, non-aromatic heterocyclic rings comprise up to three heteroatoms selected from N and O within the ring system. In yet other embodiments, non-aromatic heterocyclic rings comprise up to two heteroatoms selected from N and O within the ring system. The term includes monocyclic, bicyclic or polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl.

The term “aryl” (or “aryl ring” or “aryl group”) used alone or as part of a larger moiety as in “aralkyl”, “aralkoxy”, “aryloxyalkyl”, or “heteroaryl” refers to carbocyclic aromatic ring systems. The term “aryl” may be used interchangeably with the terms “aryl ring” or “aryl group”. “Carbocyclic aromatic ring” groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring” or “carbocyclic aromatic”, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The terms “heteroaryl”, “heteroaromatic”, “heteroaryl ring”, “heteroaryl group”, “aromatic heterocycle” or “heteroaromatic group”, used alone or as part of a larger moiety as in “heteroaralkyl” or “heteroarylalkoxy”, refer to heteroaromatic ring groups having five to fourteen members, in which one or more ring carbons is replaced by a heteroatom such as, N, S, or O. In some embodiments, heteroaryl rings comprise up to three heteroatoms selected from N, S and O within the ring. In other embodiments, heteroaryl rings comprise up to two heteroatoms selected from N, S and O within the ring system. In yet other embodiments, heteroaryl rings comprise up to three heteroatoms selected from N and O within the ring system. In yet other embodiments, heteroaryl rings comprise up to two heteroatoms selected from N and O within the ring system. Heteroaryl rings include monocyclic heteroaromatic rings and polycyclic aromatic rings in which a monocyclic aromatic ring is fused to one or more other aromatic rings. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo”, “cyclic”, “cyclic group” or “cyclic moiety”, include mono-, bi-, and tri-cyclic ring systems including cycloaliphatic, heterocycloaliphatic, aryl, or heteroaryl, each of which has been previously defined.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “bridged bicyclic ring system” refers to a bicyclic heterocycloalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “bridge” refers to a bond or an atom or an unbranched chain of atoms connecting two different parts of a molecule. The two atoms that are connected through the bridge (usually but not always, two tertiary carbon atoms) are denotated as “bridgeheads”.

As used herein, the term “spiro” refers to ring systems having one atom (usually a quaternary carbon) as the only common atom between two rings.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein an optionally substituted aralkyl can be substituted on both the alkyl and the aryl portion. Unless otherwise indicated as used herein optionally substituted aralkyl is optionally substituted on the aryl portion.

In some embodiments, an aliphatic group and a heterocyclic ring may independently contain one or more substituents. Suitable substituents on the saturated carbon of an aliphatic group or of a non-aromatic heterocyclic ring are selected from those described above. Other suitable substitutents include those listed as suitable for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, wherein each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

In some embodiments, optional substituents on the nitrogen of a heterocyclic ring include those described above. Examples of such suitable substituents include —OH, —NH₂, —NH(C₁-C₄ alkyl), —N(C₁-C₄ alkyl)₂, —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include —R⁺, —N(R⁺)₂, —C(O) R⁺, —CO₂R⁺, —C(O)C(O) R⁺, —C(O)CH₂C(O) R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

In some embodiments, an aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from those described above. Specific examples include halogen, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include: halogen; —R^{∘}; —OR^{∘}; —SR^{∘}; 1,2-methylenedioxy; 1,2-ethylenedioxy; phenyl (Ph) optionally substituted with R^{∘}; —O(Ph) optionally substituted with R^{∘}; —(CH₂)₁₋₂(Ph), optionally substituted with R^{∘}; —CH═CH(Ph), optionally substituted with R^{∘}; —NO₂; —CN; —N(R^{∘})₂; —NR^{∘}C(O)R^{∘}; —NR^{∘}C(S)R^{∘}; —NR^{∘}C(O)N(R^{∘})₂; —NR^{∘}C(S)N(R^{∘})₂; —NR^{∘}CO₂R^{∘}; —NR^{∘}NR^{∘}C(O)R^{∘}; —NR^{∘}NR^{∘}C(O)N(R^{∘})₂; —NR^{∘}NR^{∘}CO₂R^{∘}; —C(O)C(O)R^{∘}; —C(O)CH₂C(O)R^{∘}; —CO₂R^{∘}; —C(O)R^{∘}; —C(S)R^{∘}; —C(O)N(R^{∘})₂; —C(S)N(R^{∘})₂; —OC(O)N(R^{∘})₂; —OC(O)R^{∘}; —C(O)N(OR^{∘})R^{∘}; —C(NOR^{∘})R^{∘}; —S(O)₂R^{∘}; —S(O)₃R^{∘}; —SO₂N(R^{∘})₂; —S(O)R^{∘}; —NR^{∘}SO₂N(R^{∘})₂; —NR^{∘}SO₂R^{∘}; —N(OR^{∘})R^{∘}; —C(═NH)—N(R^{∘})₂; or —(CH₂)₀₋₂NHC(O)R^{∘}; wherein each independent occurrence of R^{∘} is selected from hydrogen, optionally substituted C₁₋₆ aliphatic, an unsubstituted 5-6 membered heteroaryl or heterocyclic ring, phenyl, —O(Ph), or —CH₂(Ph), or, two independent occurrences of R^{∘}, on the same substituent or different substituents, taken together with the atom(s) to which each R^{∘} group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group of R^{∘} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, CHO, N(CO)(C₁₋₄ aliphatic), C(O)N(C₁₋₄ aliphatic), wherein each of the foregoing C₁₋₄aliphatic groups of R^{∘} is unsubstituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

The term “unsaturated”, as used herein, means that a moiety has one or more units of unsaturation.

As detailed above, in some embodiments, two independent occurrences of R^{∘} (or R⁺, or any other variable similarly defined herein), may be taken together with the atom(s) to which each variable is bound to form a 5-8-membered heterocyclyl, aryl, or heteroaryl ring or a 3-8-membered cycloalkyl ring. Exemplary rings that are formed when two independent occurrences of R^{∘} (or R⁺, or any other variable similarly defined herein) are taken together with the atom(s) to which each variable is bound include, but are not limited to the following: a) two independent occurrences of R^{∘} (or R⁺, or any other variable similarly defined herein) that are bound to the same atom and are taken together with that atom to form a ring, for example, N(R^{∘})₂, where both occurrences of R^{∘} are taken together with the nitrogen atom to form a piperidin-1-yl, piperazin-1-yl, or morpholin-4-yl group; and b) two independent occurrences of R^{∘} (or R⁺, or any other variable similarly defined herein) that are bound to different atoms and are taken together with both of those atoms to form a ring, for example where a phenyl group is substituted with two occurrences of OR^{∘}

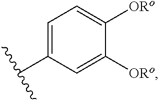

these two occurrences of R^{∘} are taken together with the oxygen atoms to which they are bound to form a fused 6-membered oxygen containing ring:

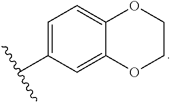 [SMILES: *c1ccc2c(c1)OCCO2]

It will be appreciated that a variety of other rings can be formed when two independent occurrences of R^{∘} (or R⁺, or any other variable similarly defined herein) are taken together with the atom(s) to which each variable is bound and that the examples detailed above are not intended to be limiting.

As used herein, an “amino” group refers to —NH₂.

The term “hydroxyl” or “hydroxy” or “alcohol moiety” refers to —OH.

As used herein, an “oxo” refers to ═O.

As used herein, the term “alkoxy”, or “alkylthio”, as used herein, refers to an alkyl group, as previously defined, attached to the molecule through an oxygen (“alkoxy” e.g., —O-alkyl) or sulfur (“alkylthio” e.g., —S-alkyl) atom.

As used herein, the terms “halogen”, “halo”, and “hal” mean F, Cl, Br, or I.

As used herein, the term “cyano” or “nitrile” refer to —CN or —C≡N.

The terms “alkoxyalkyl”, “alkoxyalkenyl”, “alkoxyaliphatic”, and “alkoxyalkoxy” mean alkyl, alkenyl, aliphatic or alkoxy, as the case may be, substituted with one or more alkoxy groups.

The terms “haloalkyl”, “haloalkenyl”, “haloaliphatic”, “haloalkoxy”, and “cyclo(haloalkyl)” mean alkyl, alkenyl, aliphatic, alkoxy, or cycloalkyl, as the case may be, substituted with one or more halogen atoms. This term includes perfluorinated alkyl groups, such as —CF₃ and —CF₂CF₃.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. In some embodiments, the cyanoalkyl is (NC)-alkyl-.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups, wherein the amino group is as defined above.

The terms “hydroxyalkyl”, “hydroxyaliphatic”, and “hydroxyalkoxy” mean alkyl, aliphatic or alkoxy, as the case may be, substituted with one or more —OH groups.

The terms “alkoxyalkyl”, “alkoxyaliphatic”, and “alkoxyalkoxy” mean alkyl, aliphatic or alkoxy, as the case may be, substituted with one or more alkoxy groups. For example, an “alkoxyalkyl” refers to an alkyl group such as (alkyl-O)-alkyl-, wherein alkyl has been defined above.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

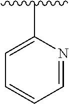 [SMILES: *c1ccccn1]

also represents

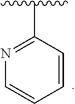 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds described herein can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by, for example, 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by, for example, 1) reacting the purified compound in its acid form with a suitable organic or inorganic base; and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds described herein, the methods of the invention can be employed for preparing pharmaceutically acceptable solvates (e.g., hydrates) and clathrates of these compounds.

As used herein, the term “pharmaceutically acceptable solvate,” is a solvate formed from the association of one or more pharmaceutically acceptable solvent molecules to one of the compounds described herein. The term solvate includes hydrates (e.g., hemihydrate, monohydrate, dihydrate, trihydrate, tetrahydrate, and the like).

As used herein, the term “hydrate” means a compound described herein or a salt thereof that further includes a stoichiometric or non-stoichiometric amount of water bound by non-covalent intermolecular forces.

As used herein, the term “clathrate” means a compound described herein or a salt thereof in the form of a crystal lattice that contains spaces (e.g., channels) that have a guest molecule (e.g., a solvent or water) trapped within.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds of the invention are provided in the form of a single enantiomer at least 95%, at least 97% and at least 99% free of the corresponding enantiomer.

In a further embodiment, the compounds of the invention are in the form of the (+) enantiomer at least 95% free of the corresponding (−) enantiomer.

In a further embodiment, the compounds of the invention are in the form of the (+) enantiomer at least 97% free of the corresponding (−) enantiomer.

In a further embodiment, the compounds of the invention are in the form of the (+) enantiomer at least 99% free of the corresponding (−) enantiomer.

In a further embodiment, the compounds of the invention are in the form of the (−) enantiomer at least 95% free of the corresponding (+) enantiomer.

In a further embodiment, the compounds of the invention are in the form of the (−) enantiomer at least 97% free of the corresponding (+) enantiomer.

In a further embodiment the compounds of the invention are in the form of the (−) enantiomer at least 99% free of the corresponding (+) enantiomer.

In some embodiments, the compounds of the invention are provided as pharmaceutically acceptable salts. As discussed above, such pharmaceutically acceptable salts can be derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄_{⁺} (where R is C₁₋₄ alkyl) salts, choline and tromethamine.

In one embodiment of the invention, the pharmaceutically acceptable salt is a sodium salt.

In one embodiment of the invention, the pharmaceutically acceptable salt is a potassium salt.

In one embodiment of the invention, the pharmaceutically acceptable salt is a lithium salt.

In one embodiment of the invention, the pharmaceutically acceptable salt is a tromethamine salt.

In one embodiment of the invention, the pharmaceutically acceptable salt is an L-arginine salt.

It will be appreciated by those skilled in the art that the compounds of the invention described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds of the invention described herein can exist in different solvate forms, for example hydrates. Solvates of the compounds of the invention may also form when solvent molecules are incorporated into the crystalline lattice structure of the compound molecule during the crystallization process.

The terms “subject,” “host,” or “patient” includes an animal and a human (e.g., male or female, for example, a child, an adolescent, or an adult). Preferably, the “subject,” “host,” or “patient” is a human.

In one embodiment, the present invention provides methods for treating or preventing a Flaviviridae viral infection in a host comprising administering to the host a therapeutically effective amount of at least one compound according to the invention described herein.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

In one embodiment, the methods of the invention are directed for treatment of HCV genotype 1 infection. In another embodiment, the HCV is genotype 1a or genotype 1b.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In treating or preventing one or more conditions/diseases described above, the compounds described above can be formulated in pharmaceutically acceptable formulations that optionally further comprise a pharmaceutically acceptable carrier, adjuvant or vehicle.

In one embodiment, the present invention provides a pharmaceutical composition comprising at least one compound according to the invention described herein and at least one pharmaceutically acceptable carrier, adjuvant, or vehicle, which includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the compounds described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the compounds described above and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds described above and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a compound according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

In one embodiment, the present invention provides a pharmaceutical composition comprising at least one compound according to the invention described herein, and further comprising one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In another embodiment, there is provided a combination therapy of at least one compound according to the invention described herein in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The additional agents for the compositions and combinations include, for example, ribavirin, amantadine, merimepodib, Levovirin, Viramidine, and maxamine.

In one combination embodiment, the compound and additional agent are administered sequentially.

In another combination embodiment, the compound and additional agent are administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121A2 (Idenix), WO 02/069903 A2 (Biocryst Pharmaceuticals Inc.), and WO 02/057287 A2 (Merck/Isis) and WO 02/057425 A2 (Merck/lsis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190 (Gilead), and PF-868,554 (Pfizer).

The term “viral NS5A inhibitor” as used herein means an agent that is effective to inhibit the function of the viral NS5A protease in a mammal Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

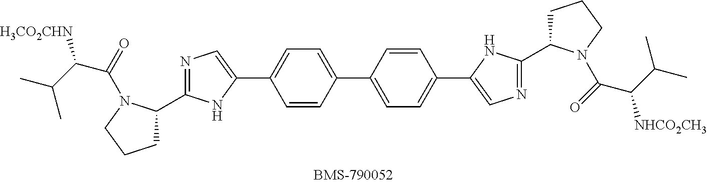

(Gao M. et al. Nature, 465, 96-100 (2010): nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class Il interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir (Schering-Plough), VX-950/telaprevir (Vertex) and ITMN-B (InterMune), GS9132 (Gilead), TMC-435350 (Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-lntron™ regimen is 1.0 mg/kg/week subcutaneously for one year. The dose should be administered on the same day of the week.

When administered in combination with ribavirin, the recommended dose of PEG-lntron is 1.5 micrograms/kg/week.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments: viral serine protease inhibitor is HCV serine protease inhibitor; viral polymerase inhibitor is HCV polymerase inhibitor; viral helicase inhibitor is HCV helicase inhibitor.

In one embodiment, the present invention provides a pharmaceutical composition comprising at least one compound according to the invention described herein, one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, and at least one pharmaceutically acceptable carrier or excipient.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein for inhibiting or reducing the activity of viral polymerase in a host.

In a further embodiment, the composition or combination according to the invention further comprises at least one compound according to the invention described herein; one or more additional agents select from non-nucleoside HCV polymerase inhibitors (e.g., HCV-796), nucleoside HCV polymerase inhibitors (e.g., R7128, R1626, R1479), and HCV NS3 protease inhibitors (e.g., VX-950/telaprevir and ITMN-191); and interferon and/or ribavirin.

In one embodiment, the additional agent is interferon α 1A, interferon α 1B, interferon α 2A, or interferon α 2B, and optionally ribavirin.

In one embodiment, the present invention provides a method for treating or preventing a HCV viral infection in a host comprising administering to the host a combined therapeutically effective amounts of at least one compound according to the invention described herein, and one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin.

In one combination embodiment, the compound and additional agent are administered sequentially.

In another combination embodiment, the compound and additional agent are administered simultaneously.

In one embodiment, there is provided a method for inhibiting or reducing the activity of HCV viral polymerase in a host comprising administering to the host a combined therapeutically effective amounts of at least one compound of the invention, and one or more additional agents select from non-nucleoside HCV polymerase inhibitors (e.g., HCV-796) and nucleoside HCV polymerase inhibitors (e.g., R7128, R1626, R1479), interferon and ribavirin.

The combinations referred to above may conveniently be presented for use in the form of a pharmaceutical formulation and thus pharmaceutical formulations or compositions comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of at least one compound of the invention, in combination with the use of one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, for the manufacture of a medicament for treating or preventing a HCV infection in a host.

When the compounds of the invention described herein are used in combination with at least one second therapeutic agent active against the same virus, the dose of each compound may be either the same as or differ from that when the compound is used alone. Appropriate doses will be readily appreciated by those skilled in the art.

The ratio of the amount of a compound according to the invention described herein administered relative to the amount of the additional agent (non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon or ribavirin) will vary dependent on the selection of the compound and additional agent.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### Synthesis of Compounds of the Invention

The compounds according to the invention described herein can be prepared by any suitable method known in the art, for example, U.S. Pat. No. 6,881,741, US 2005/0009804, US 2006/0276533, WO 2002/100851, and WO 08/58393. Preparation details of some exemplary compounds are described below. Syntheses of certain exemplary compounds of the invention are described below. Generally, the compounds of the invention can be prepared as shown in those syntheses optionally with any desired appropriate modification.

General Analytical Methods.

As used herein the term RT (min) refers to the LCMS retention time, in minutes, associated with the compound. Unless otherwise indicated, the method employed to obtain the reported retention times is as follows:

Column: YMC-Pack Pro C18, 50 mm×4.6 mm id

Gradient: 10-95% methanol/H₂O. Flow rate: 1.5 ml/min. UV-vis detection.

### General Analytical Methods and Methodology for Synthesis and Characterization of Compounds

As used herein the term RT (min) refers to the LCMS retention time, in minutes, associated with the compound. NMR and Mass Spectroscopy data of certain specific compounds are summarized in Table 1.

### 1A: Preparation of Compound 1

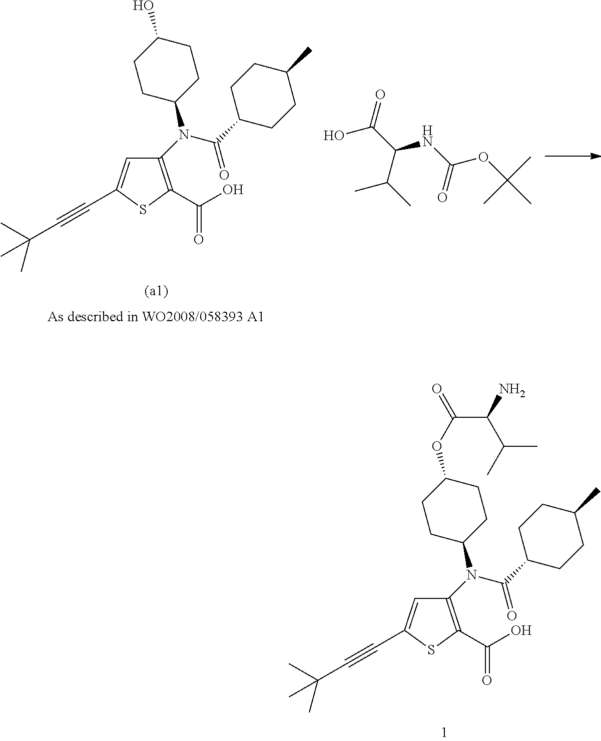 [SMILES: CC(C)(C)C#Cc1cc(N(C(=O)[C@H]2CC[C@H](C)CC2)[C@H]2CC[C@H](O)CC2)c(C(=O)O)s1 ; CC(C)[C@H](NC(=O)OC(C)(C)C)C(=O)O ; CC(C)[C@H](N)C(=O)O[C@H]1CC[C@H](N(c2cc(C#CC(C)(C)C)sc2C(=O)O)C(=O)[C@H]2CC[C@H](C)CC2)CC1]

5-(3,3-Dimethylbut-1-ynyl)-3-[(trans-4-hydroxycyclohexyl)-(4-trans methylcyclohexanecarbonyl)amino]thiophene-2-carboxylic acid (compound (al), 300 mg, 0.67 mmol) was dissolved in dichloromethane (DCM, 15 mL). To this was added (25)-2-(tert-butoxycarbonylamino)-3-methyl-butanoic acid Boc-L-valine (176 mg, 0.81 mmol), N,N-dimethylpyridin-4-amine (DMAP, 8.22 mg, 0.067 mmol), triethylamine (Et₃N, 136 mg, 187 μL, 1.35 mmol), and 3-(ethyliminomethyleneamino)-N,N-dimethyl-propan-1-amine hydrochloride (EDC, 129 mg, 0.67 mmol). The reaction was stirred overnight. The reaction mixture was then concentrated, diluted with ethyl acetate (EtOAc), washed with water, and the combined organic layers washed with brine and dried with sodium sulfate. Filtration and concentration gave a yellow oil, which was purified by column chromatography. The resulting product was then treated with 4N HCl in dioxane (15 mL) to give the desired compound 1 as the HCl salt (100 mg, 26%): MS: m/z (obs.): 545.4 [M+H]⁺; Retention time: 3.45 min; 1H NMR (300 MHz, MeOH) δ 7.04 (s, 1H), 4.75-4.58 (m, 1H), 4.39 (dt, J=14.5, 9.4 Hz, 1H), 3.85 (d, J=4.4 Hz, 1H), 3.80-3.68 (m, 1H), 3.61-3.51 (m, 1H), 2.24 (dt, J=14.0, 6.9 Hz, 1H), 2.01 (dd, J=15.2, 7.3 Hz, 6H), 1.60 (dd, J=28.5, 14.8 Hz, 9H), 1.34 (s, 9H), 1.18-0.99 (m, 3H), 0.81 (d, J=6.5 Hz, 3H), 0.66 (dd, J=25.3, 12.9 Hz, 1H).

### 1B: Preparation of Compound 2

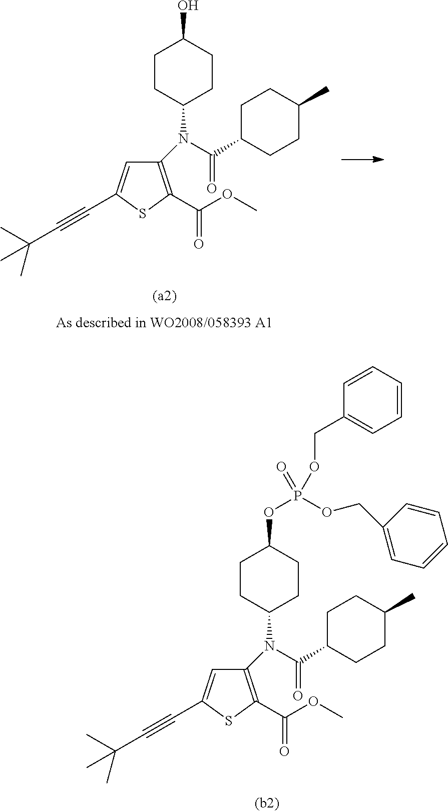

To a stirred solution of methyl 5-(3,3-dimethylbut-1-ynyl)-3-[(trans 4-hydroxycyclohexyl)-(trans 4-methylcyclohexanecarbonyl)amino]thiophene-2-carboxylate (compound (a2), 110 mg, 0.24 mmol) in dichloromethane (DCM, 4.4 mL) was added tetrazole (0.839 mg, 0.012 mmol) in acetonitrile (440 μL), followed by N-dibenzyloxyphosphanyl-N-isopropyl-propan-2-amine (108 mg, 105 μL, 0.31 mmol) and phenylisocyanate (31.4 mg, 28.6 μL, 0.26 mmol). The resulting reaction mixture was stirred for 14 h at ambient temperature, then cooled to 0° C. and hydrogen peroxide added dropwise (30% w/v solution, 1.90 mL, 16.8 mmol). The solution was stirred for 2 h, then quenched slowly with saturated aqueous sodium sulfite while the temperature was maintained at 0° C. The resulting mixture was extracted with dichloromethane (2×20 mL), the combined organic extracts were washed with brine (25 mL), dried over sodium sulfate, filtered, and concentrated under reduced pressure. The crude product (6 spots on TLC, eluent 70% ethyl acetate in hexanes) was purified by silica-gel chromatography (30-80% ethyl acetate in hexane, 12 g ISCO column) to afford the desired compound (b2)(80 mg). LC/MS data were consistent with structure, HPLC and ¹H NMR revealed mixture of products (major desired), which was taken into the next step without further purification: MS: m/z (obs.): 720.45 [M+H]⁺; Retention time: 6.20 min.

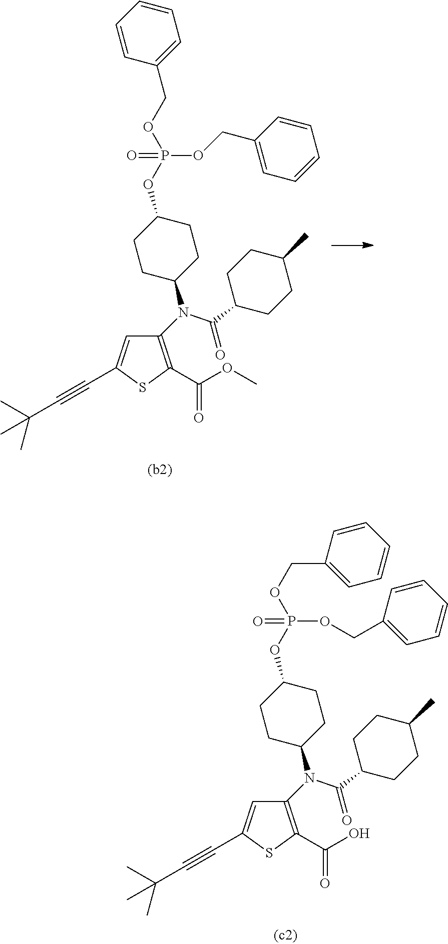 [SMILES: COC(=O)c1sc(C#CC(C)(C)C)cc1N(C(=O)[C@H]1CC[C@H](C)CC1)[C@H]1CC[C@H](OP(=O)(OCc2ccccc2)OCc2ccccc2)CC1 ; CC(C)(C)C#Cc1cc(N(C(=O)[C@H]2CC[C@H](C)CC2)[C@H]2CC[C@H](OP(=O)(OCc3ccccc3)OCc3ccccc3)CC2)c(C(=O)O)s1]

Methyl 3-[(trans (4-dibenzyloxyphosphoryloxy)cyclohexyl)-(4-trans methylcyclohexanecarbonyl)amino]-5-(3,3-dimethylbut-1-ynyl)thiophene-2-carboxylate (compound (b2), 400 mg, 0.56 mmol) was dissolved in tetrahydrofuran (THF, 20 mL), then water (5 mL) and lithium hydroxide (13.3 mg, 0.56 mmol) added. The mixture was stirred over night, after which LC/MS showed desired product. The reaction was diluted with ethyl acetate and the aqueous layer acidified with 1N HCl, extracted with ethyl acetate, and the combined organic layers dried with brine and sodium sulfate. Filtration and concentration gave a yellow oil which was purified by silica gel chromatography (ISCO instrument) to give 300 mg desired compound (c2): MS: m/z (obs.): 706.4 [M+H]⁺; Retention time: 6.28 min.

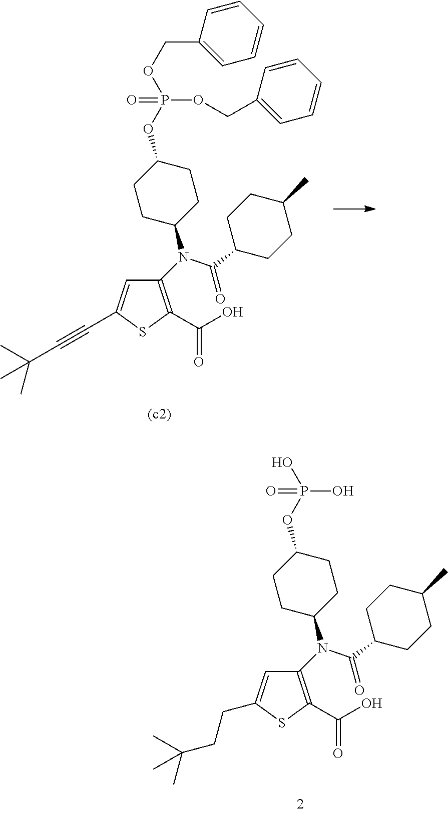 [SMILES: CC(C)(C)C#Cc1cc(N(C(=O)[C@H]2CC[C@H](C)CC2)[C@H]2CC[C@H](OP(=O)(OCc3ccccc3)OCc3ccccc3)CC2)c(C(=O)O)s1 ; CC(C)(C)CCc1cc(N(C(=O)[C@H]2CC[C@H](C)CC2)[C@H]2CC[C@H](OP(=O)(O)O)CC2)c(C(=O)O)s1]

3-[(4-trans(dibenzyloxyphosphoryloxy)cyclohexyl)-(4-trans methylcyclohexanecarbonyl)amino]-5-(3,3-dimethylbut-1-ynyl)thiophene-2-carboxylic acid (compound (c2), 300 mg, 0.425 mmol) was dissolved in methanol (MeOH, 15 mL) and treated with palladium hydroxide on carbon (Pearlman's catalyst) (60 mg), under an atmosphere of hydrogen. The reaction was stirred overnight, then filtered over celite and concentrated to give compound 2: MS: m/z (obs.): 530.4 [M+H]⁺; Retention time: 7.04 min; ¹H NMR (300 MHz, d6-DMSO) δ 6.77 (s, 1H), 4.27 (d, J=6.8 Hz, 1H), 4.03 (dd, J=14.2, 7.1 Hz, 1H), 3.69 (s, 1H), 2.79 (dd, J=9.1, 5.2 Hz, 2H), 1.89 (d, J=10.3 Hz, 3H), 1.79 (s, 1H), 1.70-1.47 (m, 7H), 1.44-1.11 (m, 7H), 0.93 (s, 9H), 0.82 (d, J=6.7 Hz, 1H), 0.75 (d, J=6.4 Hz, 3H).

### 1C: Preparation of Compound 3

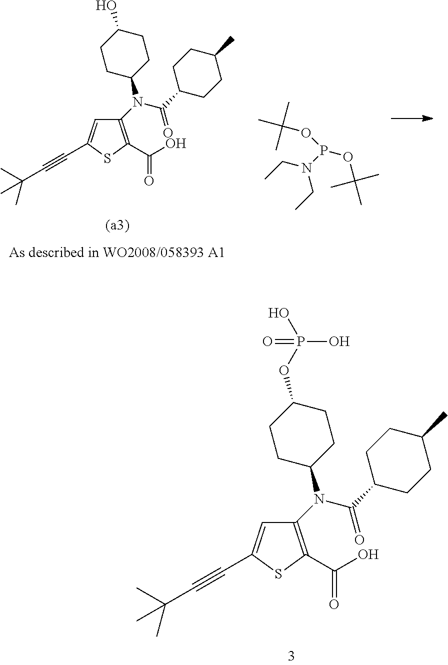 [SMILES: CC(C)(C)C#Cc1cc(N(C(=O)[C@H]2CC[C@H](C)CC2)[C@H]2CC[C@H](O)CC2)c(C(=O)O)s1 ; CCN(CC)P(OC(C)(C)C)OC(C)(C)C ; CC(C)(C)C#Cc1cc(N(C(=O)[C@H]2CC[C@H](C)CC2)[C@H]2CC[C@H](OP(=O)(O)O)CC2)c(C(=O)O)s1]

5-(3,3-Dimethylbut-1-ynyl)-3-[(trans 4-hydroxycyclohexyl)-(trans 4-methylcyclohexanecarbonyl)amino]thiophene-2-carboxylic acid (compound (a3), 100 mg, 0.12 mmol) was dissolved in dichloromethane (DCM, 10.0 mL) and cooled to 0° C. Tetrazole (4.0 mg, 0.058 mmol) was added followed by N-(di-tert-butoxyphosphanyl)-N-ethyl-ethanamine (288 mg, 322 μL, 1.16 mmol). The reaction was stirred overnight at room temperature, then cooled to −78° C. 3-Chlorobenzenecarboperoxoic acid (MCPBA) (99.7 mg, 0.58 mmol) was added and the reaction stirred for 2 hours then quenched with aq. Na₂SO₃. The mixture was extracted with ethyl acetate and the extracts washed with water. The organic layer was concentrated to give a colorless oil, which was purified by ISCO silica gel chromatography and taken directly to the next step. To the product was added CH₂Cl₂ (5 mL) and 2,2,2-trifluoroacetic acid (TFA) (5 mL). The reaction was stirred for 2 hours, then concentrated and the product 3 purified by HPLC: MS: m/z (obs.): 526.39 [M+H]⁺; Retention time: 6.51 min; ¹H NMR (300 MHz, d6-DMSO) δ 7.18 (s, 1H), 4.29 (t, J=11.8 Hz, 1H), 3.83 (s, 1H), 2.53 (d, J=8.2 Hz, 3H), 1.84 (s, 2H), 1.75-1.33 (m, 7H), 1.30 (s, 9H), 1.27-1.09 (m, 3H), 0.90 (d, J=12.9 Hz, 2H), 0.76 (d, J=6.5 Hz, 2H), 0.70-0.47 (m, 2H); ³¹P NMR (121.5 MHz, d6-DMSO) δ −2.01 (s).

### 1D: Preparation of Compound 4

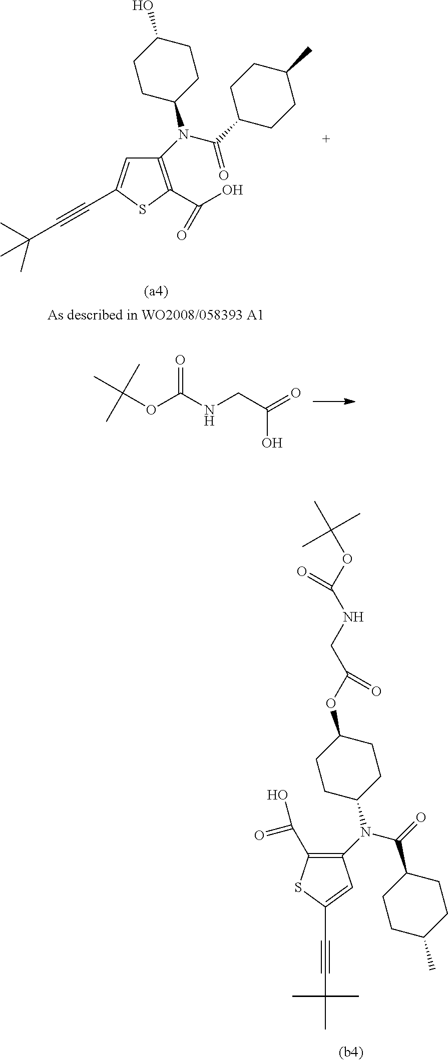

To a solution of 5-(3,3-dimethylbut-1-ynyl)-3-[(4-trans-hydroxycyclohexyl)-(4-trans-methylcyclohexanecarbonyl)amino]thiophene-2-carboxylic acid (compound (a4), 75 mg, 0.17 mmol) and N-Boc-glycine (44.2 mg, 0.25 mmol) in CH₂Cl₂ (15 mL) was added 3-(ethyliminomethyleneamino)-N,N-dimethyl-propan-1-amine hydrochloride (EDC) (32.2 mg, 0.17 mmol), N,N-dimethylpyridin-4-amine (DMAP) (10.3 mg, 0.084 mmol) and Et₃N (34 mg, 0.33 mmol). The reaction mixture was stirred at ambient temperature overnight then the reaction mixture was evaporated and purified by ISCO silica gel chromatography to give compound (b4), [O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid: MS: m/z (obs.): 603.17 [M+H]⁺; Retention time: 2.31 min.

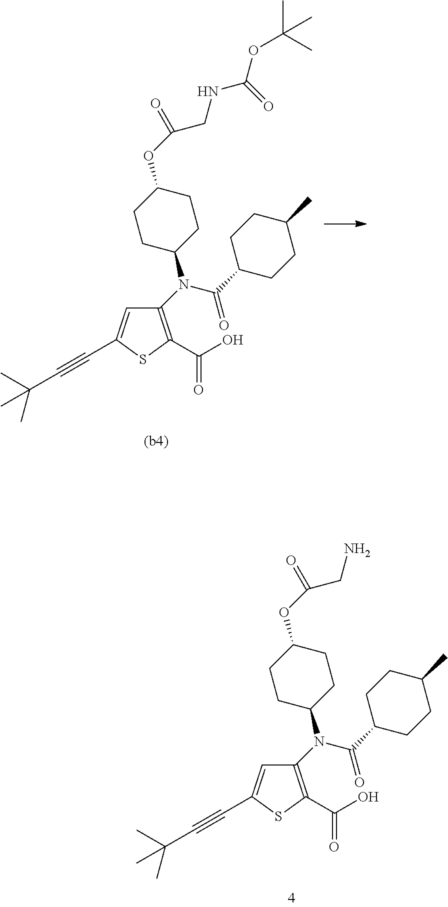

[O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid (Compound (b4), 40 mg, 0.066 mmol) was treated with 4N HCl in dioxane (1 mL) and stirred at RT overnight. Then the reaction mixture was concentrated and purified by HPLC to give compound 4 (11 mg): MS: m/z (obs.): 503.35 [M+H]⁺; Retention time: 2.24 min.

### 1E: Preparation of Compound 5

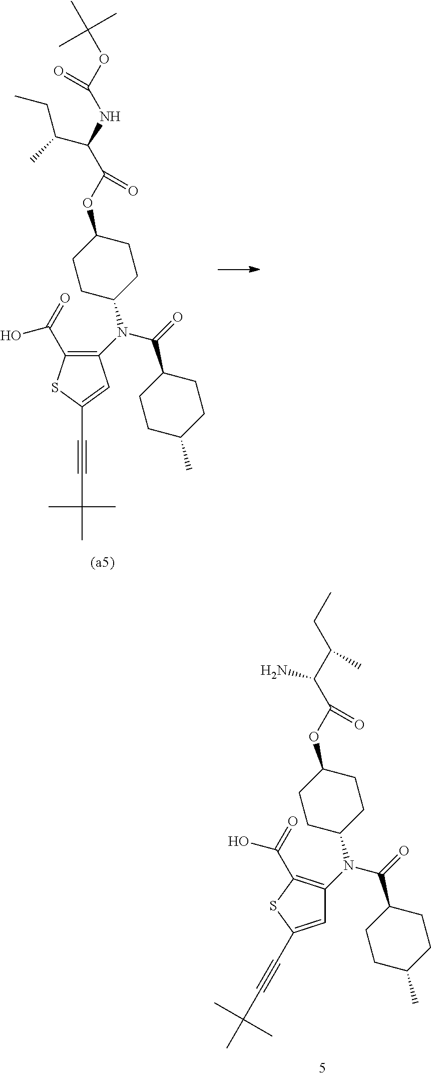 [SMILES: CC[C@@H](C)[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a5), [O—(N-t-Butoxycarbonyl)-D-isoleucyl]-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-isoleucine as described for Compounds 1 & 4 above) was treated with 4N HCl in dioxane (10 mL) and stirred at RT overnight. Then the reaction mixture was concentrated and purified by HPLC to give compound 5: MS: m/z (obs.): 559.4 [M+H]⁺; Retention time: 2.39 min.

### 1F: Preparation of Compound 6

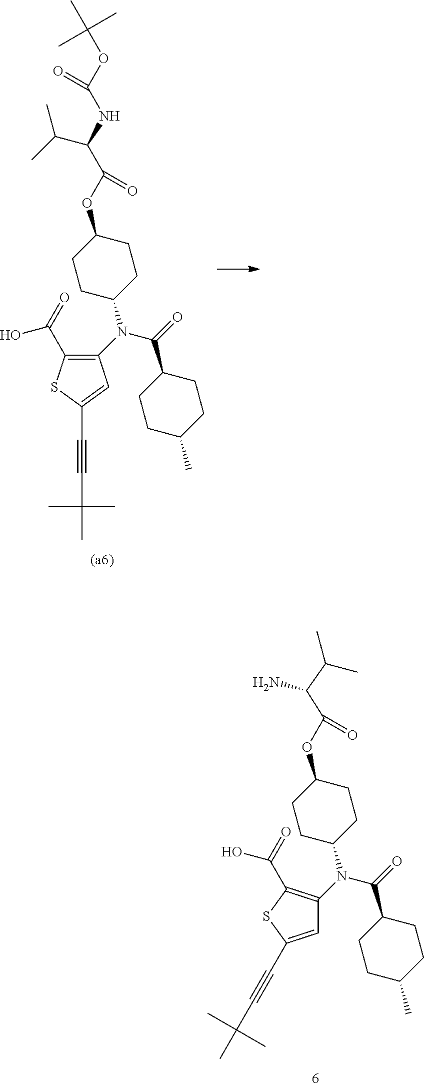 [SMILES: CC(C)[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC(C)[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a6), [O—(N-t-Butoxycarbonyl)-D-valinyl]-5-(3,3-dimethylbut-1-ynyl)-3-[(4-trans-hydroxycyclohexyl)-(4-trans-methylcyclohexanecarbonyl)amino]thiophene-2-carboxylic acid (30 mg) (prepared from Boc-D-valine as described for Compounds 1 & 4 above) was treated with 4N HCl in dioxane (10 mL) and stirred at RT overnight. Then the reaction mixture was concentrated and purified by HPLC to give compound 6: MS: m/z (obs.): 545.39 [M+H]⁺; Retention time: 2.35 min.

### 1G: Preparation of Compound 7

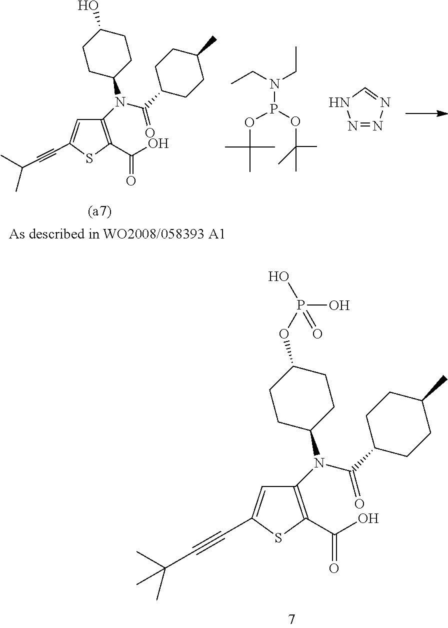

3-[(4-Hydroxycyclohexyl)-(4-methylcyclohexanecarbonyl)amino]-5-(3-methylbut-1-ynyl)thiophene-2-carboxylic acid (compound (a7), 400 mg, 0.93 mmol) was dissolved in CH₂Cl₂ (20 mL) and tetrazole (32 mg, 0.46 mmol) added. Then the reaction mixture was cooled to 0° C. and N-di-tert-butoxyphosphanyl-N-ethyl-ethanamine added (2.31 g, 2.58 mL, 9.27 mmol). Then the reaction mixture was stirred at RT overnight. Then again cooled to −78° C., and hydrogen peroxide added (31.5 mg, 28.4 μL, 0.93 mmol). The reaction mixture was stirred for 2 hours, quenched with aq. Na₂SO₃ and diluted with water and ethyl acetate. The organic layer was washed with brine and dried with Na₂SO₄, then concentrated to give an oil. This was purified by ISCO silica gel chromatography and taken directly to the next step. To the product was added CH₂Cl₂ (5 mL) and 2,2,2-trifluoroacetic acid (TFA) (5 mL). The reaction was stirred for 2 hours, then concentrated. Repeated evaporation from HCl/Et₂O afforded the solid product 7: MS: m/z (obs.): 512.18 [M+H]⁺; Retention time: 2.98 min.

### 1G: Preparation of Compound 8

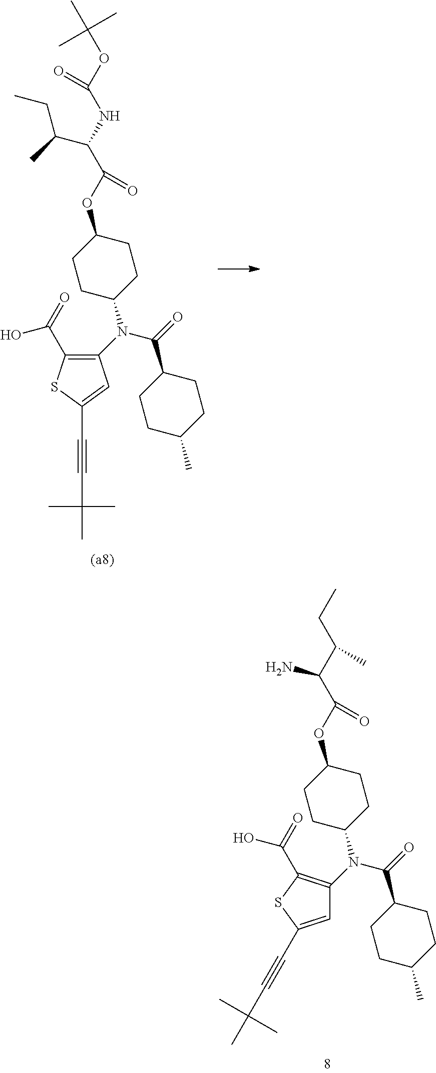 [SMILES: CC[C@H](C)[C@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@H](N)C(=O)O[C@H]1CC[C@H](N(c2cc(C#CC(C)(C)C)sc2C(=O)O)C(=O)[C@H]2CC[C@H](C)CC2)CC1]

Compound (a8), (O—(N-t-Butoxycarbonyl)-L-iso leucyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-isoleucine as described for Compounds 1 & 4 above) (35 mg) was treated with 4N HCl in dioxane (10 mL) and stirred at RT for overnight. Then the reaction mixture was concentrated and purified by HPLC to give compound 8: MS: m/z (obs.): 559.47 [M+H]⁺; Retention time: 3.2 min.

### 1H: Preparation of Compound 9

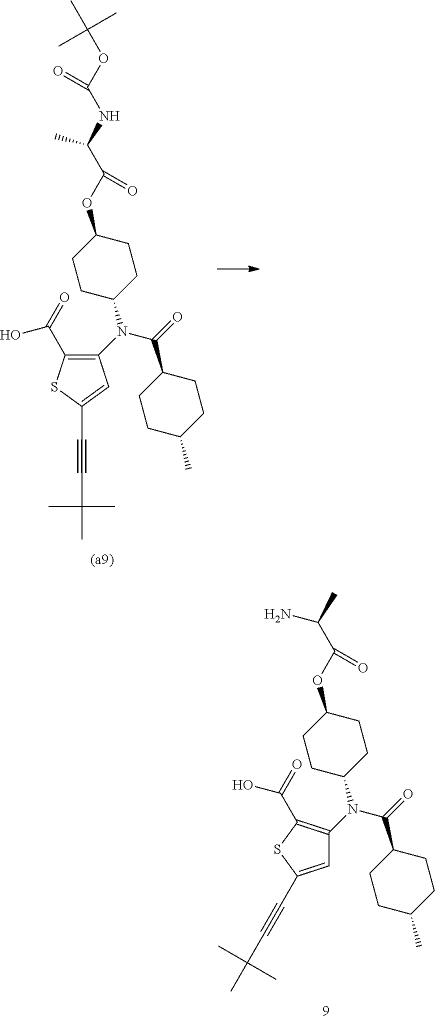

Compound (a9), (O—(N-t-Butoxycarbonyl)-L-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-alanine as described for Compounds 1 & 4 above) (25 mg) was taken in 4N HCl in dioxane and stirred at RT overnight. Then the reaction mixture was concentrated and purified by HPLC to give compound 9: MS: m/z (obs.): 517.43 [M+H]⁺ Retention time: 2.99 min.

### 1I: Preparation of Compound 10

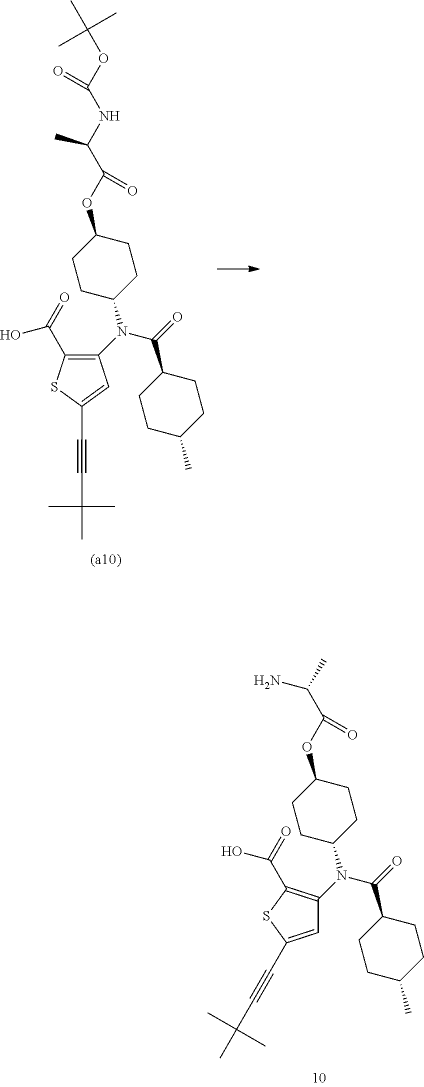 [SMILES: C[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; C[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a10), (O—(N-t-Butoxycarbonyl)-D-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-alanine as described for Compounds 1 & 4 above) (35 mg, 0.058 mmol) was treated with 4N HCl in dioxane (10 mL) and stirred at RT overnight. Then the reaction mixture was concentrated and purified by HPLC to give compound 10: MS: m/z (obs.): 517.43 [M+H]⁺; Retention time: 3.0 min.

### 1.1: Preparation of Compound 11

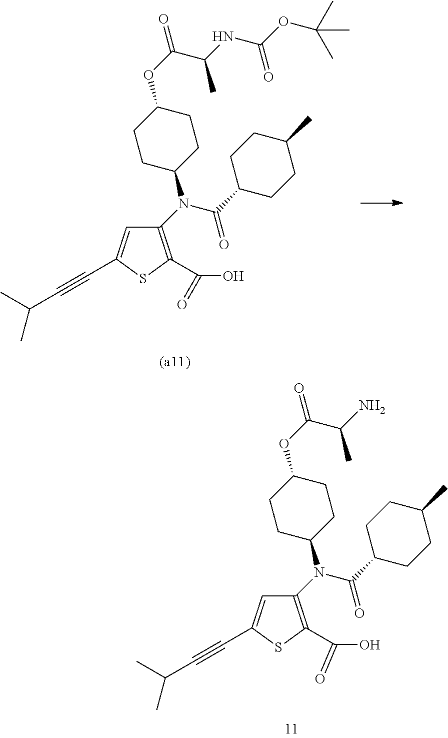

Compound (a11), (O—(N-t-Butoxycarbonyl)-L-alanyl)-5-(3-methylbut-1-ynyl)-3-[(4-trans-hydroxycyclohexyl)-(4-trans-methylcyclohexanecarbonyl)amino]thiophene-2-carboxylic acid (prepared from Boc-L-alanine as described for Compounds 1 & 4 above) (900 mg, 1.493 mmol) was treated with 4N HCl in dioxane (10 mL). The reaction was stirred overnight, then concentrated. Diethyl ether was added to precipitate the desired product 11: ¹H NMR (300 MHz, DMSO) δ 8.31 (s, 2H), 7.22 (s, 1H), 4.70-4.49 (m, 1H), 4.36 (t, J=11.7 Hz, 1H), 3.48 (d, J=4.1 Hz, 1H), 1.97-1.65 (m, 2H), 2.0 (m, 16H), 1.36 (dd, J=7.1, 1.4 Hz, 3H), 1.23 (d, J=6.9 Hz, 6H), 1.07-0.92 (m, 1H), 0.76 (d, J=6.4 Hz, 3H), 0.60 (dd, J=22.6, 10.7 Hz, 1H); MS: m/z (obs.): 503.54 [M+H]⁺; Retention time: 2.22 min.

TABLE 1
LCMS and NMR data of the compounds described in FIGs.1 and 2

| | LCMS | LCMS | |
|---|---|---|---|
| Compounds | [M + H]⁺ | RT | NMR |
| 1 | 545.45 | 3.45 | 1 H NMR (300 MHz, MeOH) δ 7.04 (s, 1 H), 4.75 − 4.58 (m, 1 H), 4.39 |
| | | | (dt, J =14.5, 9.4 Hz, 1 H), 3.85 (d, J = 4.4 Hz, 1 H), 3.80 − 3.68 (m, |
| | | | 1 H), 3.61 − 3.51 (m, 1 H), 2.24 (dt, J = 14.0, 6.9 Hz, 1 H), 2.01 (dd, J = |
| | | | 15.2, 7.3 Hz, 6H), 1.60 (dd, J = 28.5, 14.8 Hz, 9H), 1.34 (s, 9H), 1.18− |
| | | | 0.99 (m, 3H), 0.81 (d, J = 6.5 Hz, 3H), 0.66 (dd, J = 25.3, 12.9 Hz, |
| | | | 1 H). |
| 2 | 530.35 | 7.04 | ¹ H NMR (300 MHz, d6−DMSO) δ 6.77 (s, 1 H), 4.27 (d, J = 6.8 Hz, |
| | | | 1 H), 4.03 (dd, J = 14.2, 7.1 Hz, 1 H), 3.69 (s, 1 H), 2.79 (dd, J = 9.1, |
| | | | 5.2 Hz, 2H), 1.89 (d, J = 10.3 Hz, 3H), 1.79 (s, 1 H), 1.70 − 1.47 (m, |
| | | | 7H), 1.44 − 1.11 (m, 7H), 0.93 (s, 9H), 0.82 (d, J = 6.7 Hz, 1 H), 0.75 |
| | | | (d, J = 6.4 Hz, 3H). |
| 3 | 526.39 | 6.51 | ¹ H NMR (300 MHz, d6−DMSO) δ 7.18 (s, 1 H), 4.29 (t, J = 11.8 Hz, |
| | | | 1 H), 3.83 (s, 1 H), 2.53 (d, J = 8.2 Hz, 3H), 1.84 (s, 2H), 1.75 − 1.33 |
| | | | (m, 7H), 1.30 (s, 9H), 1.27 − 1.09 (m, 3H), 0.90 (d, J = 12.9 Hz, 2H), |
| | | | 0.76 (d, J = 6.5 Hz, 2H), 0.70 − 0.47 (m, 2H); ³¹P NMR (121.5 MHz, |
| | | | d6−DMSO) δ −2.01 (s). |
| 4 | 503.35 | 2.24 | |
| 5 | 559.4 | 2.39 | |
| 6 | 545.39 | 2.35 | |
| 7 | 512.18 | 2.98 | |
| 8 | 559.47 | 3.2 | |
| 9 | 517.43 | 2.99 | |
| 10 | 517.43 | 3 | |
| 11 | 503.54 | 2.22 | ¹H NMR (300 MHz, DMSO) δ 8.31 (s, 2H), 7.22 (s, 1 H), 4.70 − 4.49 |
| | | | (m, 1 H), 4.36 (t, J = 11.7 Hz, 1 H), 3.48 (d, J = 4.1 Hz, 1 H), 1.97 − |
| | | | 1.65 (m, 2H), 2.0 (m, 16H), 1.36 (dd, J = 7.1, 1.4 Hz, 3H), 1.23 (d, J = |
| | | | 6.9 Hz, 6H), 1.07 − 0.92 (m, 1 H), 0.76 (d, J = 6.4 Hz, 3H), 0.60 (dd, |
| | | | J = 22.6, 10.7 Hz, 1 H) |

### Example 2

### PK Parameters of Prodrugs of the Invention

The prodrug whose PK parameters are to be determined can be formulated as a solution in 0.5% MC/0.5% Tween 80/99% water and administered orally by gavage to rats at a dose of 3 mg/kg. Rats are weighed the day before the study. Rat plasma is sampled predose and at 15, 30 min, 1, 2, 3, 4, 6, 8, 12 and 24 hrs post dose using Instech automatic blood sampling equipment. Blood is collected in tubes containing K2-EDTA and 110 uL plasma are extracted for analysis. Rats are fed ad lib and standard IACUC and SOP protocols are followed. Plasma samples and dose samples are analyzed using LC/MS/MS for both the prodrug compound and the active metabolite. PK parameters for both analytes for each subject are calculated using the measured dose of prodrug.

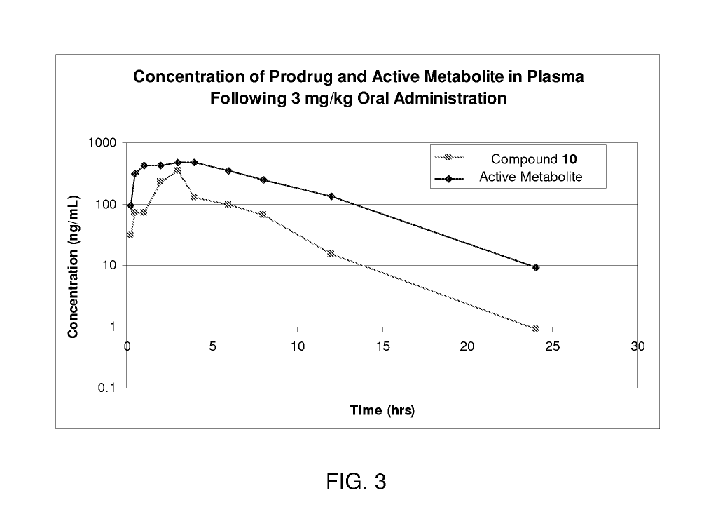

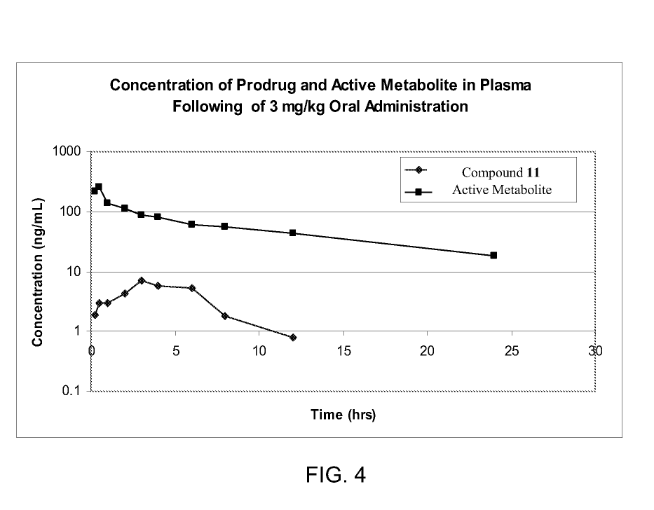

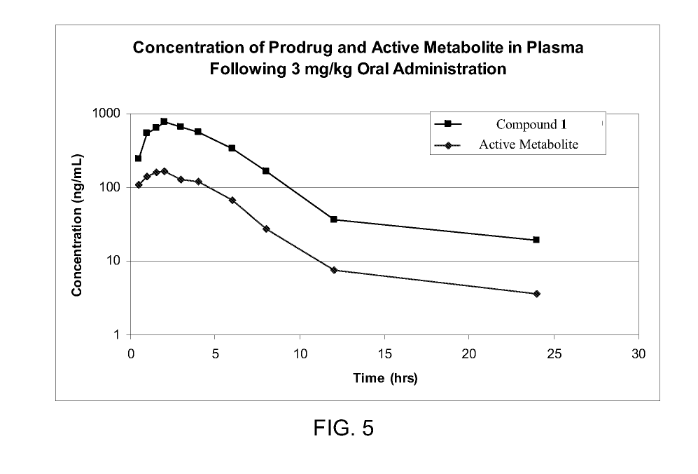

The PK parameters of a prodrug of the invention (compound 10) was measured as described in the preceding paragraph, and are depicted in FIG. 3. As shown in FIG. 3, the —O-alaninyl group of compound 10 in vivo converted into the —OH active metabolite. Similarly, the PK parameters of compound 11 and 1, which were measured as described in the preceding paragraph, are also depicted in FIGS. 4 and 5, respectively. As shown in FIGS. 4 and 5, compounds 11 and 1 were in vivo converted into their active metabolites.

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound represented by Structural Formula (I): 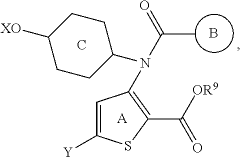
or a pharmaceutically acceptable salt thereof, wherein:
Ring A is optionally further substituted with one or more substituents selected from the group consisting of halogen, -D, —CD₃, —CHD₂, —CH₂D, —CN, C₁₋₄ alkyl, and C₁₋₄ haloalkyl;
Ring B is a C₃₋₈ cycloalkyl ring or a 5-6 membered aryl ring, each of which optionally and independently is substituted with one or more substituents selected from the group consisting of halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂;
Ring C is a cyclohexyl ring optionally further substituted with one or more substituents selected from the group consisting of halogen, -D, —CD₃, —CHD₂, —CH₂D, C₁₋₄ alkyl, C₁₋₄ haloalkyl, —OH, —O(C₁₋₄ alkyl), —CN, —NH₂, —NH(C₁₋₄ alkyl), and —N(C₁₋₄ alkyl)₂;
X is [—C(O)—C(R⁴R⁵)—N(R)—]ₙ—C(O)C(R⁶R²R⁸), —P(O)(OR³)₂, or —C(O)R²;
Y is C₃₋₈ carbocycle, 5-8 membered heterocycle, —(C₂ aliphatic group)-R¹, C₆₋₁₀ aryl, or 5-10 membered heteroaryl, wherein each of said carbocycle, heterocycle, aryl and heteroaryl is optionally and independently substituted with one or more instances of J^{Y} independently selected from the group consisting of halogen, —CN, nitro, azido, R^{a}, —SO₂R^{a}, —OR^{a}, —COR^{a}, —NRR^{a}, —C(O)OR^{a}, —OC(O)R^{a}, —NRC(O)R^{a}, —C(O)NRR^{a}, —NRC(O)NRR^{a}, —NRC(O)OR^{a}, —OCONRR^{a}, —SO₂NRR^{a}, —NRSO₂R^{a}, —NRSO₂NRR^{a}, and —NRC(═NR)NRR^{a}, and wherein said C₂ aliphatic group is optionally substituted with one or more substitutents selected from the group consisting of halogen, —CN, C₁₋₂ alkyl, C₁₋₂ haloalkyl, hydroxy, and methoxy;
R¹ is i) —H; ii) a C₁₋₆ aliphatic group optionally substituted with one or more instances of J^{1A}; iii) a C₃₋₁₀ carbocycle or 4-10 membered heterocycle, each of which is optionally and independently substituted with one or more instances of J^{1B}; or iv) a C₆₋₁₀ aryl or 5-10 membered heteroaryl group, each of which is optionally and independently substituted with one or more instances of J^{1C};
R² is: i) a C₃₋₁₀ carbocyclic or 4-10 membered heterocyclic group, each of which is optionally and independently substituted with one or more instances of J^{E}, or ii) a C₆₋₁₀ aryl or 5-10 membered heteroaryl group, each of which is optionally and independently substituted with one or more instances of J^{F};
R³ is: i) —H, ii) a C₁₋₆ aliphatic group optionally substituted with one or more instances of J^{D}, iii) a C₃₋₁₀ carbocyclic or 4-10 membered heterocyclic group, each of which is optionally and independently substituted with one or more instances of J^{E}, or iv) a C₆₋₁₀ aryl or 5-10 membered heteroaryl group, each of which is optionally and independently substituted with one or more instances of J^{F};
each of R⁴, R⁵, R⁶, and R⁷ independently is —H; or C₁₋₆ alkyl optionally substituted with one or more substitutents selected from the group consisting of —OH, —O(C₁₋₆ alkyl), —NH₂, —NH(C₁₋₆ alkyl), —N(C₁₋₄ alkyl)₂, —NHC(═NH)NH₂, NHC(═NH)NH(C₁₋₆ alkyl), NHC(═NH)N(C₁₋₆ alkyl)₂, —CO₂H, —CO₂(C₁₋₆ alkyl), —C(O)NH₂, —C(O)NH(C₁₋₆ alkyl), —C(O)N(C₁₋₆ alkyl)₂, —NHC(O)(C₁₋₆ alkyl), phenyl, hydroxyphenyl, imidazole, and indole;
R⁸ is —R^{b}, halogen, cyano, nitro, —OR^{b}, —NR^{b}R^{c}, —C(O)R^{b}, —C(O)OR^{b}, —OC(O)R^{b}, —NRC(O)R^{b}, or —C(O)NR^{b}R^{c}; or
optionally when R⁸ is —NR^{b}R^{c}, R^{c} and R⁷ form a pyrrolidine ring; and
R⁹ is: i) —H; ii) a C₁₋₆ aliphatic group optionally substituted with one or more instances of J^{9A}; iii) a C₃₋₁₀ carbocycle or 4-10 membered heterocycle, each of which is optionally and independently substituted with one or more instances of J^{9B}; or iv) a C₆₋₁₀ aryl or 5-10 membered heteroaryl group, each of which is optionally and independently substituted with one or more instances of J^{9C};
each of J^{1A} and J^{9A} independently is oxo or Q; or two J^{1A} and two J^{9A}, respectively, together with the atom(s) to which they are attached, optionally and independently form a 3-8-membered non-aromatic ring that is optionally substituted with one or more instances of J^{E};
each of J^{1B} and J^{9B} and independently is oxo, Q, or a C₁₋₆ aliphatic group optionally substituted with one or more instances of Q; or two J^{1B} and two J^{9B}, respectively, together with the atom(s) to which they are attached, optionally and independently form a 3-8-membered non-aromatic ring that is optionally substituted with one or more instances of J^{E};
each of J^{1C} and J^{9C} independently is Q or a C₁₋₆ aliphatic group optionally substituted with one or more instances of Q; or two J^{1C} and two J^{9C}, respectively, together with the atoms to which they are attached, optionally and independently form a 3-8-membered non-aromatic ring that is optionally substituted with one or more instances of J^{E};
each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —OC(O)OR^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F};
each R^{a}, R^{b}, and R^{e} independently is: i) —H; ii) a C₁₋₆ aliphatic group optionally substituted with one or more substituents independently selected from the group consisting of halogen, oxo, nitro, —CN, —OR′, —NR′R′, —OCOR′, —COR″, —CO₂R′, —CONR′R′, —NR′C(O)R′, C₃₋₈ carbocyclic group optionally substituted with one or more instances of J^{E}, 4-8 membered heterocyclic group optionally substituted with one or more instances of J^{E}, C₆₋₁₀ aryl group optionally substituted with one or more instances of J^{F}, and 5-10 membered heteroaryl group optionally substituted with one or more instances of J^{F}; iii) a C₃₋₈ carbocyclic or 4-8 membered heterocyclic group, each of which is optionally and independently substituted with one or more instances of J^{E}; or iv) a C₆₋₁₀ aryl or 5-10 membered heteroaryl group, each of which is optionally and independently substituted with one or more instances of J^{F}; or
R^{a}, together with R and the nitrogen atom to which it is attached, optionally forms a 4-8 membered heterocycle optionally substituted with one or more instances of J^{E}; or
R^{b} and R^{c}, together with the nitrogen atom to which they are attached, optionally forms a 4-8 membered heterocycle optionally substituted with one or more instances of J^{E}; and
each R is independently —H or a C₁₋₆ aliphatic group optionally substituted with one or more instances of J^{D};
each R′ is independently —H or a C₁₋₆ aliphatic group optionally substituted with one or more instances of J^{D}; or two R′, together with the nitrogen atom to which they are attached, optionally form a 4-8 membered heterocycle optionally substituted with one or more instances of J^{E};
each R″ is a C₁₋₆ aliphatic group optionally substituted with one or more instances of J^{D};
each J^{D} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl;
each J^{E} is independently selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), and C₁-C₆ aliphatic group optionally substituted with one or more instances of J^{D};
each J^{F} is independently selected from the group consisting of halogen, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), and C₁-C₆ aliphatic that is optionally substituted with one or more instances of J^{D}; and
n is 0 or 1.

2. The compound of claim 1, wherein:
each Q independently is selected from the group consisting of halogen, cyano, nitro, —OR^{a}, —SR^{a}, —S(O)R^{a}, —SO₂R^{a}, —NRR^{a}, —C(O)R^{a}, —C(O)OR^{a}, —OC(O)R^{a}, —NRC(O)R^{a}, —C(O)NRR^{a}, —NRC(O)NRR^{a}, —NRC(O)OR^{a}, —NRC(═NR)NRR^{a}, —OCONRR^{a}, —C(O)NRC(O)OR^{a}, —C(═NR)R^{a}, —C(═NOR)R^{a}, —SO₂NRR^{a}, —NRSO₂R^{a}, —NRSO₂NRR^{a}, —OP(O)(OR^{a})OR^{a}, C₃₋₈ carbocycle optionally substituted with one or more instances of J^{E}, 4-8 membered heterocycle optionally substituted with one or more instances of J^{E}, C₆₋₁₀ aryl group optionally substituted with one or more instances of J^{F}, and 5-10 membered heteroaryl group optionally substituted with one or more instances of J^{F};
Y is optionally substituted C₃₋₆ cycloalkyl, optionally substituted C₄₋₆ cycloalkenyl, —(C₂ aliphatic group)-R¹, optionally substituted phenyl, or optionally substituted 5-6 membered heteroaryl, and wherein said C₂ aliphatic group is optionally substituted; and
each J^{Y} is independently selected from the group consisting of halogen, —CN, nitro, R^{a}, —OR^{a}, —COR^{a}, and —NRR^{a}.

3-12. (canceled)

13. The compound of claim 2, wherein Y is —(C₂ aliphatic group)-R¹ or phenyl optionally substituted with one or more instances of J^{Y} independently selected from the group consisting of chloro, fluoro, —CN, nitro, methyl, ethyl, —CF₃, —OH, —OMe, —NH₂, and —C(O)Me, and wherein said C₂ aliphatic group is optionally substituted.

14. (canceled)

15. The compound of claim 13, wherein Y is —CH₂—CH₂—R¹, —CH═CH—R¹, or —C≡CR¹.

16. The compound of claim 15, represented by Structural Formula (II): 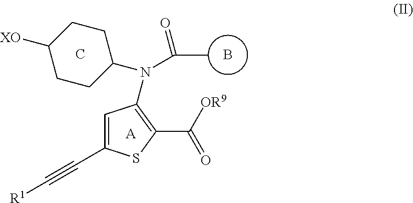
or a pharmaceutically acceptable salt thereof, wherein:
each of rings A, B and C is independently and optionally substituted;
X is [—C(O)—C(R⁴R⁵)—N(R)—]ₙ—C(O)C(R⁶R⁷R⁸) or —P(O)(OR³)₂;
R¹ is an optionally substituted C₁₋₆ alkyl or C₃₋₈cycloalkyl group, each of which is optionally and independently substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl;
each R³ independently is —H, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl;
each of R⁴, R⁵, R⁶, and R⁷ independently is —H; or C₁₋₆ alkyl optionally substituted with one or more substitutents selected from the group consisting of —OH, —NH₂, —NHC(═NH)NH₂, —CO₂H, —C(O)NH₂, phenyl, hydroxyphenyl, imidazole, and indole;
R⁸ independently is —H, halogen, cyano, —OR^{b}, —NR^{b}R^{c}, optionally substituted C₁-C₆ aliphatic, optionally substituted C₃₋₆ carbocyclic, optionally substituted 4-8 membered heterocyclic, optionally substituted phenyl, or optionally substituted 5-6 remembered heteroaryl; and
R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —OCOO(C₁-C₆ alkyl), —CO(C₁-C₆ alkyl), —CO₂H, —CO₂(C₁-C₆ alkyl), —O(C₁-C₆ alkyl), —O(C₁-C₆haloalkyl), C₃₋₇ cycloalkyl, C₃₋₇ cyclo(haloalkyl), phenyl, and 5-6 membered heterocycle optionally substituted with one or more substituents selected from the group consisting of oxo and C₁-C₆ alkyl.

17-22. (canceled)

23. The compound of claim 16, wherein:
each R³ independently is —H or optionally substituted C₁₋₆ alkyl;
R⁴ and R⁶ are each independently —H or C₁₋₆ alkyl; and
R⁵ and R⁷ are each independently —H or optionally substituted C₁₋₆ alkyl.

24. The compound of claim 23, wherein R⁸ is —NR^{b}R^{c}.

25. (canceled)

26. (canceled)

27. The compound of claim 24, wherein:
R³ is —H or C₁₋₆ alkyl optionally substituted with one or more substituents selected from the group consisting of halogen, —CN, —OH, —O(C₁-C₆ alkyl), C₃₋₇ cycloalkyl, and phenyl;
each of R⁴ and R⁶ is independently —H or C₁₋₆ alkyl;
each of R⁵ and R⁷ independently is —H or C₁₋₆ alkyl optionally substituted with one or more substitutents selected from the group consisting of —OH, —NH₂, —NH—C(═NH)—NH₂, —CO₂H, and —C(O)NH₂; and
each of R^{b} and R^{c} independently is —H or C₁₋₆ alkyl optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), —O(C₁₋₆ haloalkyl), C₃₋₇ cycloalkyl, C₃₋₇ cyclo(haloalkyl), and phenyl, or R^{b} and R^{c}, together with the nitrogen atom to which they are attached, optionally form a 5-7 membered heterocyclic ring optionally substituted with one or more substituents selected from the group consisting of halogen, oxo, —CN, —OH, —NH₂, —NH(C₁₋₆ alkyl), —N(C₁₋₆ alkyl)₂, —OCO(C₁₋₆ alkyl), —CO(C₁₋₆ alkyl), —CO₂H, —CO₂(C₁₋₆ alkyl), —O(C₁₋₆ alkyl), and —O(C₁₋₆ haloalkyl).

28-30. (canceled)

31. The compound of claim 27, wherein:
each of R⁴ and R⁶ is independently —H;
each of R^{b} and R^{c} independently is —H or C₁₋₆ alkyl, or optionally, together with the nitrogen atom to which they are attached, form an optionally substituted, 5-7 membered, heterocyclic ring;
ring B is optionally substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂; and
ring C is optionally further substituted with one or more substituents selected from the group consisting of halogen, -D, —CD₃, —CHD₂, —CH₂D, —CH₃, —CF₃, —OH, —O(CH₃), —CN, —NH₂, —NH(CH₃), and —N(CH₃)₂.

32-34. (canceled)

35. The compound of claim 31, represented by Structural Formula (III) or (IV): 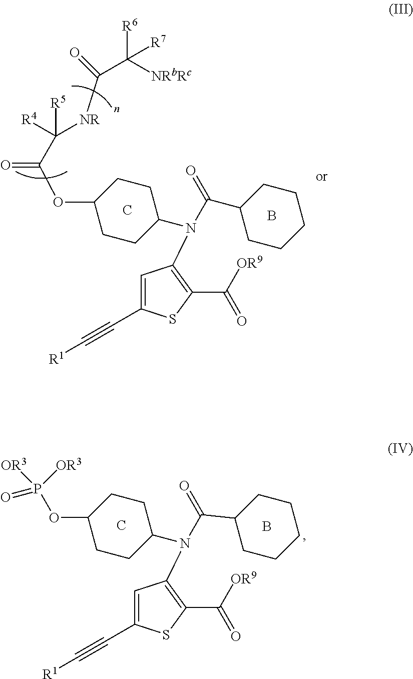
or a pharmaceutically acceptable salt thereof, wherein each ring B is optionally substituted cyclohexyl, and ring C is optionally further substituted.

36. (canceled)

37. The compound of claim 35, represented by Structural Formula (VI) or (VII), 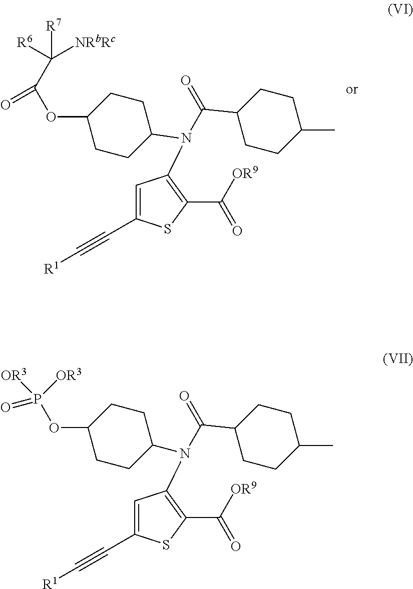
or a pharmaceutically acceptable salt thereof, wherein:
R¹ is C₁₋₆ alkyl or C₃₋₈ cycloalkyl, each of which optionally and independently substituted with one or more substituents selected from the group consisting of halogen, —CN, —OH, —O(C₁₋₆alkyl), and —O(C₁₋₆ haloalkyl);
each R³ independently is —H or C₁₋₆ alkyl;
R⁶ is —H;
R⁷ independently is —H or C₁₋₆ alkyl optionally substituted with one or more substitutents selected from the group consisting of —OH, —NH₂, —NH—C(═NH)—NH₂, —CO₂H, and —C(O)NH₂;
R⁹ is —H or C₁₋₆ alkyl optionally substituted with one or more substituents selected from the group consisting of halogen, oxo, —CN, —OH, —NH₂, —NH(C₁-C₆ alkyl), —N(C₁-C₆ alkyl)₂, —OCO(C₁-C₆ alkyl), —CO(C₁-C₆ alkyl), —CO₂H, —CO₂(C₁-C₆ alkyl), —O(C₁-C₆ alkyl), —O(C₁-C₆ haloalkyl), C₃₋₇ cycloalkyl, C₃₋₇ cyclo(haloalkyl), and phenyl; and
R^{b} and R^{c} independently is —H or C₁₋₆ alkyl.

38-44. (canceled)

45. The compound of claim 37, wherein R¹ is C₁₋₆ alkyl or C₃₋₈ cycloalkyl.

46. (canceled)

47. The compound of claim 45, wherein R¹ is t-butyl or isopropyl.

48-50. (canceled)

51. The compound of claim 47, wherein R⁹ is —H.

52. (canceled)

53. A compound represented by any one of the following structural formulae, or a pharmaceutically acceptable salt thereof

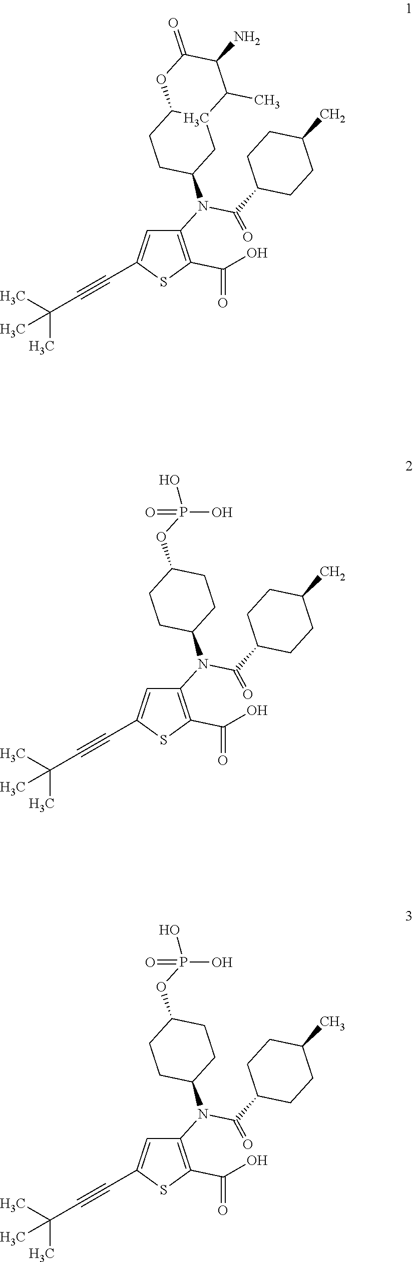

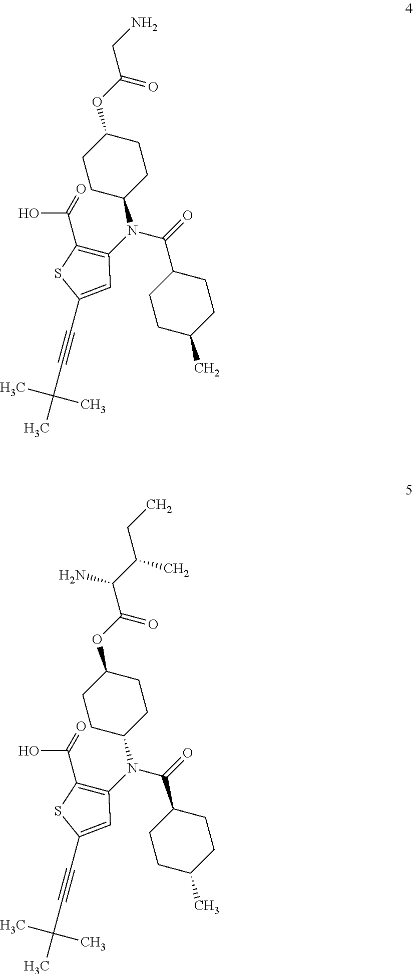

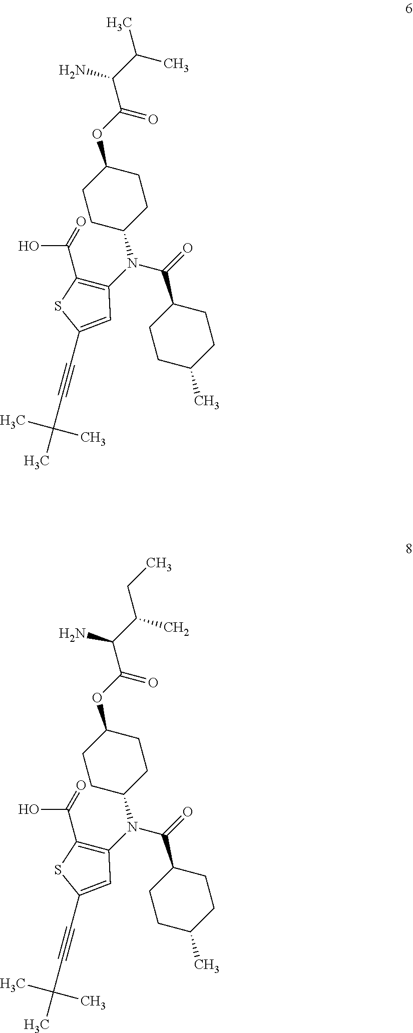 [SMILES: CC(C)[C@@H](N)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@H](N)C(=O)O[C@H]1CC[C@H](N(c2cc(C#CC(C)(C)C)sc2C(=O)O)C(=O)[C@H]2CC[C@H](C)CC2)CC1]

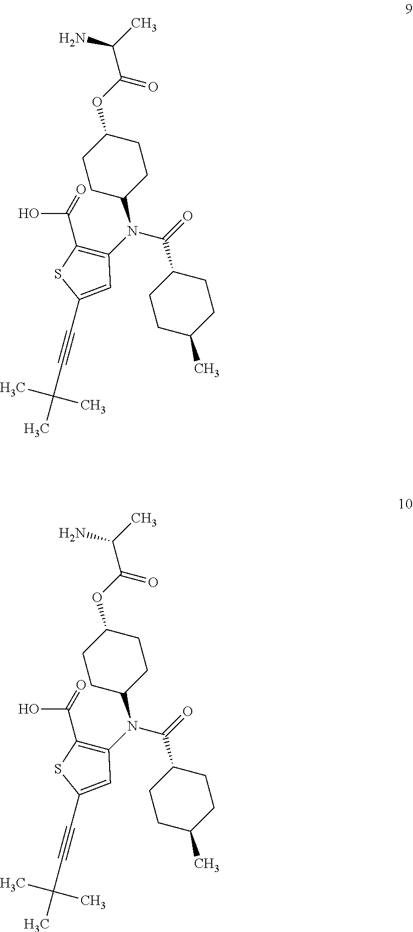

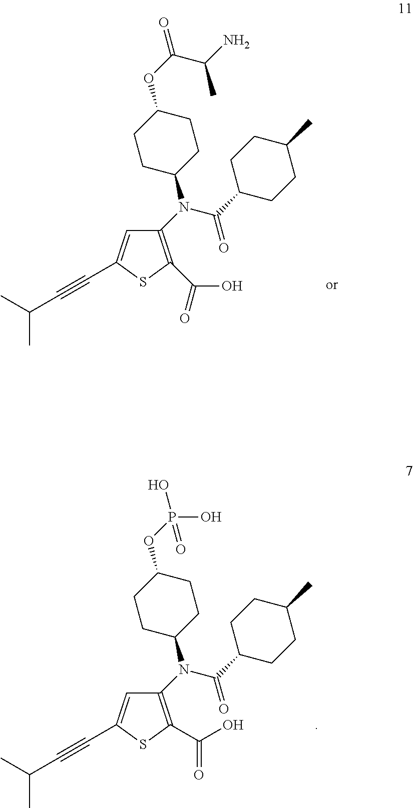

54. (canceled)

55. A pharmaceutical composition, comprising a compound of claim 1, and a pharmaceutically acceptable carrier or excipient.

56. A method of inhibiting or reducing the activity of HCV polymerase in a biological in vitro sample, comprising administering to the sample an effective amount of a compound of claim 1.

57. A method of treating a HCV infection in a subject, comprising administering to the subject a therapeutically effective amount of a compound of claim 1.

58. A method of inhibiting or reducing the activity of HCV polymerase in a subject, comprising administering to the subject a therapeutically effective amount of a compound of claim 1.

59-70. (canceled)

71. A method of preparing a compound represented by Structural Formula (I) or a pharmaceutically acceptable salt thereof, wherein the variables of Structural Formula (1) are each and independently as described in claim 1, and wherein the method comprises the step of reacting compound (1j) with X—OH for X=—[C(O)C(R⁴R⁵)N(R)]ₙC(O)C(R⁶R⁷)NR^{b}R^{c} or X=—(CO)R², or with (R^{k})₂N—P(OR³)₂ for X=—P(O)(OR³)₂ to form a compound of Structural Formula (I): 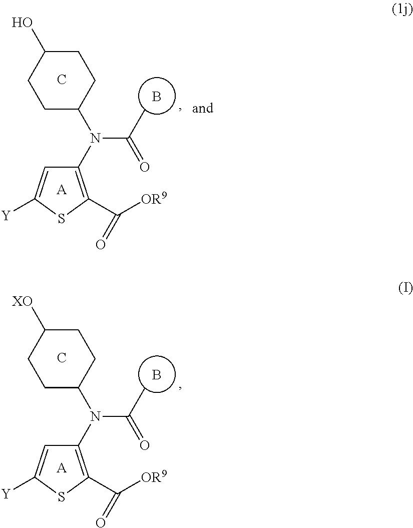
wherein:
the variables of compound (1j) are each and independently as described for Structural Formula (I); and
R^{k} is —H, C₁₋₆ alkyl or benzyl.

72. A method of preparing a compound represented by Structural Formula (II) or a pharmaceutically acceptable salt thereof, wherein the variables of Structural Formula (1) are each and independently as described in claim 1, and wherein the method comprises the step of reacting compound (2j) with X—OH for X=—[C(O)C(R⁴R⁵)N(R)]ₙC(O)C(R⁶R⁷)NR^{b}R^{c} and X=—(CO)R², or with (R^{k})₂N—P(OR³)₂ for X=—P(O)(OR³)₂ to form a compound of Structural Formula (II): 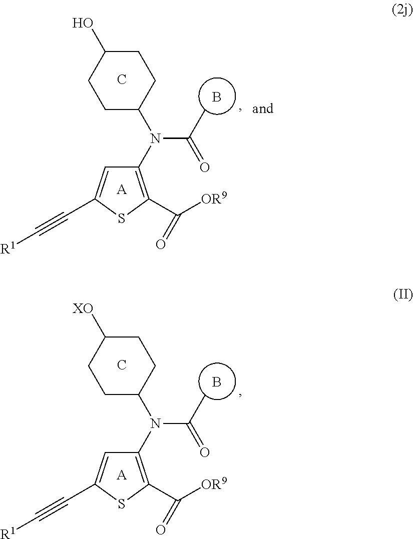
wherein:
the variables of compound (1j) are each and independently as described for Structural Formula (II); and
R^{k} is —H, C₁₋₆ alkyl or benzyl.

73-74. (canceled)
